(19) United States
(12) Patent Application Publication
(10) Pub. No.: US 2005/0228041 A1
(43) Pub. Date: Oct. 13, 2005

(54) Protective and therapeutic uses for tocotrienols

(75) Inventors: Chadan Sen, New Albany, OH (US); Sashwati Roy, New Albany, OH (US); Savita Khanna, New Albany, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 10/914,339

(22) Filed: Aug. 9, 2004

Related U.S. Application Data
(60) Provisional application No. 60/493,761, filed on Aug. 8, 2003.

(51) Int. Cl. A61K 31/355

(52) U.S. Cl. 514/458

(57) ABSTRACT

Therapeutic and prophylactic agents comprising tocotrienols, and methods of using the same are provided for the treatment of and the prevention of the onset of stroke and other disorders and diseases associated with elevated glutamate levels, and the effects of lipoxygenases such as the enzyme 12-lipoxygenase.

### PRIORITY CLAIM

This application claims priority to U.S. Provisional Patent Application 60/493,761, filed Aug. 8, 2003, which is incorporated herein by reference in its entirety.

### STATEMENT ON FEDERALLY FUNDED RESEARCH

This work was supported, at least in part, by a grant from the National Institutes of Neurological Disorders and Stroke of the National Institutes of Health, R01—NS42617. The government has certain rights in this invention.

### BACKGROUND

It is believed that the onset and damage occasioned by stroke and other forms of trauma is mediated by the activity of the enzyme 12-lipoxygenase (“12-LOX”). It is likely that 12-LOX is responsible for neuronal death as a result of focal injury due to trauma, and diffuse injury due to Parkinson's, Amyotrophic Lateral Sclerosis, epilepsy, and related conditions. The activity of 12-LOX may also be responsible for development of disease in tissues other than the brain; for example, certain skin cancers, such as melanoma; cardiac damage due to cardiac trauma; and muscle degeneration and other disorders associated with HIV infection. Thus, 12-LOX is a potential target for inhibitory agents to prevent or treat diseases and disorders associated with glutamate-induced cytotoxicity. Agents and methods for targeting and inhibiting the activity of 12-LOX are desirable.

Melanoma and other cancers are regulated by complex cellular and biochemical mechanisms. Lipoxygenases have been identified as having central involvement in certain cancers. 12-Lipoxygenase (12-LOX), through its metabolite 12( )-hydroxyeicosatetraenoic acid [12( )-HETE], has been demonstrated to play a pivotal role in experimental melanoma invasion and metastasis, and 12-LOX expression may be important in early human melanoma carcinogenesis. 12-LOX expression was studied during the progression of melanoma from human melanocytic cells to benign and dysplastic naevi to malignant metastatic disease. 12-LOX expression was determined to be low in normal human skin melanocytes and increased expression was observed in melanocytes found in compound naevi, dysplastic naevi and melanomas. Melanomas had higher levels of 12-LOX expression compared with dysplastic naevi, and 12-LOX expression was significantly different between compound naevus and dysplastic naevus melanocytes. These data suggest that 12-LOX may be an important novel marker for cancer progression within the melanoma system, and therefore could be a useful biomarker and therapeutic target for melanoma chemoprevention.

Lipoxygenases, including 12-LOX, have also been implicated in cardiac cell death that results from trauma, including neurological and cardiac trauma. Generation of arachidonic acid by the ubiquitously expressed cytosolic phospholipase A(2) (PLA(2)) has a fundamental role in the regulation of cellular homeostasis, inflammation and tumorigenesis. 12-LOX catalyzes the conversion of arachidonic acid (C20:4) to 12-hydroperoxyeicosatetraenoic acid, which in turn reduces to 12-hydroxyeicosatetraenoic acid (12-HETE) by glutathione peroxidase. Activation of 12-LOX has been implicated in various pathologies of heart. Accordingly, a therapeutic agent that has an inhibitory effect on 12-LOX is desirable for the treatment of cancers and other disorders and diseases involving 12-LOX.

### SUMMARY

In accordance with the present invention, therapeutic and prophylactic methods are provided for the treatment of and the prevention of the onset of stroke and other disorders and diseases associated with the activity of lipoxygenases, such as the enzyme 12-lipoxygenase. Also in accordance with the present invention, methods for specifically enhancing the concentrations of tocotrienols in the fetal and neonatal brain are provided. Also in accordance with the present invention, methods of improving or restoring fertility are provided.

Methods for inhibiting 12-lipoxygenase mediated cytotoxicity in a subject are provided, the methods comprising; administering to a subject who is at risk for the development of 12-lipoxygenase mediated cell damage biologically effective amount of tocotrienol. Biologically effective amounts of tocotrienol inhibit the activity of 12-lipoxygenase. The methods are directed to protecting against 12-lipoxygenase mediated cell damage is selected from the group consisting of neuronal damage, cardiac tissue damage, integument damage, development of cancer such as melanoma, and muscle tissue damage.

Also in accordance with the present invention are methods for treating a subject who has suffered from neurological trauma, comprising; administering to said subject a biologically effective amount of tocotrienol. The methods are particularly useful for treating trauma such as stroke and cardiac trauma.

Also in accordance with the present invention are methods for preventing the development of melanoma in a subject at risk of developing the same, comprising; administering to said subject a biologically effective amount of tocotrienol.

Also in accordance with the present invention are regimens for the prophylaxis and treatment of cancer, comprising administering to a subject in need of the same a pharmaceutical formulation comprising tocotrienol and a pharmaceutically acceptable carrier. Individuals or subjects in need of such treatment are considered to be at risk for development of cancer due to environmental exposure such as to the sun, or other predispositions to developing cancer, or have been diagnosed with cancer.

Also in accordance with the present invention are methods for protecting neurons in a fetus comprising the step of administering to a pregnant woman who is gestating said fetus a composition comprising at least one tocotrienol.

Also in accordance with the present invention are methods for enhancing the concentration of tocotrienol in human fetal brain by administering to a pregnant woman a composition comprising at least one tocotrienol wherein said composition is substantially free of tocopherol.

Also in accordance with the present invention are methods for enhancing the concentration of tocotrienol in the brain of an human infant. In some embodiments the methods comprise administering a composition comprising at least one tocotrienol to a lactating woman and feeding to the infant the milk produced by said lactating woman.

Also in accordance with the present invention are methods for enhancing the concentration of tocotrienol in the brain of an adult human subject comprising administering to the subject a composition comprising at least one tocotrienol, wherein the composition is substantially free of tocopherol and wherein the composition is administered in the absence of foods or dietary supplements containing tocopherol. In preferred embodiments the mixture is administered at least one half hours after and at least one half hours before said human ingests foods or food supplements containing tocopherol. Good results have been obtained using the dietary supplement Tocomin.

Also in accordance with the present invention are methods for improving fertility in an animal in need of the same comprising administering to said animal a clinically effective amount of at least one tocotrienol on a daily basis. Preferably, the tocotrienol is administered daily for a period from 2 weeks to about 16 weeks prior to an intended conception. More preferably, tocotrienol is administered on a daily basis for at least 6 to 8 weeks prior to an intended conception As used in accordance with the methods of the present invention, tocotrienol compositions are administered to subjects, as needed, on a daily basis in single or multiple doses from about 1 to about 1000 mg per dose. Preferably the doses for adults are about 600 mg and are administered from 1 to 3 times per day. A preferred mode of administration is orally in the form of gel caps. The tocotrienols used according to the methods are selected from the group consisting of a-tocotrienol, β-tocotrienol, γ-tocotrienol, δ-tocotrienol, derivatives of these, and mixtures of one or more of these. In some embodiments, the compositions according to the present methods are substantially free of tocopherol.

Also in accordance with the present invention are methods for restoring fertility to an animal lacking a functional tocopherol transport protein comprising administering to said animal a formulation comprising tocotrienol.

Also in accordance with the present invention are methods for maintaining neurons in primary culture, comprising: providing at least one neuron isolated from an animal; providing culture media comprising at least one tocotrienol; and maintaining said at least one neuron in a vessel containing the culture media.

Also in accordance with the present invention are culture compositions in the form of culture media for maintaining a neuron in primary culture wherein such compositions comprise at least one tocotrienol.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DESCRIPTION OF THE DRAWINGS

The accompanying drawings, which are incorporated in and constitute a part of this specification, illustrate several embodiments of the invention, and together with the description, serve to explain the principles of the invention.

FIG. 1 shows protection against loss of neuronal viability by α-tocotrienol.

FIG. 2 shows Imaging of glutamate-induced degeneration of rat primary cortical neurons and protection by α-tocotrienol and BL15.

FIG. 3 shows pharmacologic inhibition of 12-lipoxygenase confers protection against glutamate-induced death of HT4 as well as primary immature cortical neurons (B-D).

FIG. 4 shows primary immature cortical neurons isolated from 12-lipoxygenase knock out mice are resistant to glutamate-induced death.

FIG. 5 shows products of 12-lipoxygenase activity in glutamate-treated neurons.

FIG. 6 shows the effects of 12-Lipoxygenase: over-expression, localization and sensitivity to α-tocotrienol.

FIG. 7 shows Three-dimensional modeling of 12-lipoxygenase and α-tocotrienol docking analysis.

FIG. 8 shows tocotrienol protection of cardiac cells from activity of 12-LOX.

FIG. 9 Vitamin E levels in fetal and mother rat brains.

FIG. 10 Range of the average fold changes of differentially expressed genes in E+ and E⁻ groups.

FIG. 11 Cluster image illustrating the genes differentially expressed in fetal brains of E⁺ group.

FIG. 12 Genes up-regulated in fetal brains of E⁺ group

FIG. 13 Genes down-regulated in fetal brains of E⁺ group FIG. 14 RT-PCR validation of GeneChip microarray expression analysis.

### DETAILED DESCRIPTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The term “treatment” as used herein with reference to a disease is used broadly and is not limited to a method of curing the disease. The term “treatment” includes any method that serves to reduce one or more of the pathological effects or symptoms of a disease or to reduce the onset or the rate of progression of one or more of such pathological effects or symptoms.

As used herein, the term “vitamin E” refers generically to all tocopherols and tocotrienols, including tocopherols and their derivatives having the biological activity of RRR-α-tocopherol. In nature, eight substances have been found to have vitamin E activity: α-, β, γ and δ-tocopherol; and α-, β, γ and δ tocotrienol. Often, the term vitamin E is synonymously used with α-tocopherol, although this references is more limited than the intended use of the term vitamin E herein. While D-α-tocopherol (RRR-α-tocopherol) has the highest bioavailability and represents the standard against which all the others are commonly compared, it is only one out of eight natural forms of vitamin E. Tocotrienols, formerly known as ζ, ε or η-tocopherols, are chemically similar to tocopherols except that they have an isoprenoid tail that is unsaturated at three positions, in contrast to the saturated phytyl tail common to α-, β, γ and δ-tocopherol. While α-, β, γ and δ tocopherol are predominantly found in corn, soybean and olive oils, tocotrienols are particularly rich in palm, rice bran and barley oils.

As used herein, the term “tocotrienols” refers to alpha-tocotrienols, beta-tocotrienols, gamma-tocotrienols, and delta-tocotrienols, which were formerly known as and are sometimes alternately known as ζ, ε or η-tocopherols. Tocotrienols are highly labile under oxidative conditions, and thus it is desirable to maintain tocotrienol preparations under low oxygen conditions and to avoid heating. Optimally, tocotrienols for use in pharmaceutical or dietary supplements should be maintained in evacuated dosage units.

Tocotrienols occur largely in palm oil, rice bran oil and barley. While synthetic and natural tocopherols are readily available in the market, natural tocotrienols supply is limited. Crude palm oil which is rich in tocotrienols (800-1500 ppm) offers a potential source of natural tocotrienols. Carotech, Malaysia is the only industrial plant in the world that is able to extract and concentrate tocotrienols from crude palm, oil whereby the use of innovative and novel technology, without the use of solvents maximize the extraction rate with no adverse environmental impact.

Another potential source of tocotrienols is from rice bran oil and its fatty acid distillate. However, unlike crude palm oil, the tocotrienol content in rice bran is very much lower, at about 45-55% of the total vitamin E (tocopherol & tocotrienol) content. The remaining vitamin E is made up of tocopherol. Palm oil is unique in that it contains relatively large concentration of the tocotrienol which accounts for about 75-80% of the total vitamin E. Epidemiological studies have shown that tocopherols especially alpha-tocopherol at high concentration attenuates the cholesterol-suppresive action of the tocotrienols. A such, in order to have the optimal impact of tocotrienols in reducing blood total cholesterol, preparation with low content of tocopherols (<30% of the total vitamin E) is preferred.

Dietary tocotrienols have been shown to be incorporated into circulating human lipoproteins where they react with peroxyl radicals as efficiently as the corresponding tocopherol isomers (Suarna et al., 1993; Serbinova and Packer, 1994). Dietary supplementation with tocotrienol beneficially influences the course of carotid atherosclerosis in humans (Tomeo et al., 1995). Micromolar amounts of tocotrienol, not tocopherol, have been shown to suppress the activity of hydroxy-3 methylglutaryl coenzyme A reductase, a key hepatic enzyme responsible for cholesterol synthesis (Pearce et al., 1992; Pearce et al., 1994). Tocotrienol has been shown to have superior antioxidant, free radical scavenging effects as compared to tocopherol, perhaps due to better distribution of tocotrienols in the fatty layers of the cell membrane. While tocotrienols have shown better beneficial effects than α-tocopherol, little is known about the exact mechanism of action.

Study results that have been reported prior to the disclosure of the instant invention have shown that the transport, tissue concentration, and relative biologic function of tocopherol and tocotrienol appear somewhat disparate and possibly unrelated (see Proc Soc Exp Biol Med (1993 March) 202(3):353-9). Alpha-Tocopherol transfer protein (alphaTTP), a product of the gene which causes familial isolated vitamin E deficiency, plays an important role in determining the plasma vitamin E level. Examination of the structural characteristics of vitamin E analogs, including tocotrienols, required for recognition by alphaTTP has been reported in which ligand specificity was assessed by evaluating the competition of non-labeled vitamin E analogs and alpha-[3H]tocopherol for transfer between membranes in vitro (see FEBS Lett 1997 Jun. 2; 409(1):105-8). The relative affinities of alpha TTP for the various vitamin E analogs were determined based on the degree of competition with the highest affinity measured for various forms of tocopherol and relatively low affinity for tocotrienol. It was shown that there was a linear relationship between the relative affinity and the known biological activity obtained from the rat resorption-gestation assay. These results indicate that the affinity of vitamin E analogs for alphaTTP is one of the critical determinants of biological activity. Based on the foregoing, as well as other information known in the art regarding the activities of tocotrienols and tocopherols, there is evidence that these forms of vitamin E have different functions and are involved in different biologic mechanistic systems.

Inhibition of 12-Lipoxygenase

Results from the efforts of Applicants as described herein have identified the 12-lipoxygenase (LOX) pathway as being sensitive to tocotrienol. Applicants present the first evidence demonstrating that the glutamate-induced 12-LOX activity is sensitive to nanomolar concentration of tocotrienol. Additionally, Applicants show that 12-LOX deficient primary cortical neurons are resistant to glutamate challenge.

As disclosed herein, 12 LOX, which is implicated in stroke, is effectively inhibited by tocotrienol, and the neurodegenerative effects of 12-LOX activity can be reversed if tocotrienol is administered prior to cell death. Tocotrienols can be used for protection against the damaging effects of focal and diffuse traumas, including stroke, Parkinson's, ALS, epilepsy, and other neuordegenrative disorders and traumas. At concentrations well within the physiologically relevant range, α-tocotrienol exhibits potent neuroprotective properties in HT4 as well as immature primary cortical neurons. Current results confirm a central role of 12-LOX in executing glutamate-induced oxidative toxicity of neurons and offer α-tocotrienol as a promising tool in nutrition-based therapeutics.

Dosage and Administration

The tocotrienol composition is administered to subjects who have suffered or at risk of suffering stroke and other neurological injury, whether focal or diffuse, as a result of trauma, epilepsy, ALS, Parkinsons, and other traumas and disorders. The tocotrienol compositions are administered in a an amount sufficient to achieve reversal of damage and protection from further 12-LOX mediated damage, preferably before the onset of trauma, and on a continual basis. The tocotrienol compositions are administered to adult subjects in the range from about 1 mg to 1000 mg at a frequency of about 2-3 doses per day. More preferably, dosages are provided to adult subjects in the range from about 100 mg to 800 mg at a frequency of about 2-3 doses per day. Most preferably, dosages of about 600 mg are provided to adult subjects at a frequency of about 2-3 doses per day. The preferred form of delivery is gel caps for oral ingestion. Dosages for juvenile subjects are in the range from about 1-1000 mg per dose, and more preferably in the range from about 50-500 mg per dose, and most preferably 300 mg per dose, at a frequency from about 1 to 3 doses per day.

Prevention and Treatment of Melanoma and other Cancers

12-LOX has been directly implicated in melanoma and certain other cancers. Tocotrienol is disclosed herein to have an inhibitory effect on the function of 12-LOX. Thus, tocotrienols are indicated as pharmaceutical agents for treatment of melanoma and other cancers.

Dosage and Administration

The tocotrienol composition is administered to subjects who have suffered or at risk of suffering melanoma and other cancers, particularly those involving lipoxygenases such as 12-lipoxygenase. The tocotrienol compositions are administered in an amount sufficient to achieve reversal of damage and protection from further 12-LOX mediated damage, preferably before the onset of trauma, and on a continual basis. The tocotrienol compositions are administered to adult subjects in the range from about 1 mg to 1000 mg at a frequency of about 2-3 doses per day. More preferably, dosages are provided to adult subjects in the range from about 100 mg to 800 mg at a frequency of about 2-3 doses per day. Most preferably, dosages of about 600 mg are provided to adult subjects at a frequency of about 2-3 doses per day. The preferred form of delivery is gel caps for oral ingestion. Dosages for juvenile subjects are in the range from about 1-1000 mg per dose, and more preferably in the range from about 50-500 mg per dose, and most preferably 300 mg per dose, at a frequency from about 1 to 3 doses per day.

Protection of Cardiac Tissue

Dosage and Administration

The tocotrienol composition is administered to subjects who have suffered or at risk of suffering cardiac and other disorders involving lipoxygenases such as 12-lipoxygenase. The tocotrienol compositions are administered to adult subjects in the range from about 1 mg to 1000 mg at a frequency of about 2-3 doses per day. More preferably, dosages are provided to adult subjects in the range from about 100 mg to 800 mg at a frequency of about 2-3 doses per day. Most preferably, dosages of about 600 mg are provided to adult subjects at a frequency of about 2-3 doses per day. The preferred form of delivery is gel caps for oral ingestion. Dosages for juvenile subjects are in the range from about 1-1000 mg per dose, and more preferably in the range from about 50-500 mg per dose, and most preferably 300 mg per dose, at a frequency from about 1 to 3 doses per day.

Protection of Fetal, Neonatal, and Adult Brain Tissue

Vitamin E in the form of tocopherol is known to improve the status of fetuses and neonates in connection with certain forms of cellular damage. Recent scientific evidence has demonstrated that tocotrienol forms of Vitamin E have cellular effects that are different from those of tocopherols; in neuronal cells, tocotrienols have been shown to be more potent than tocopherols in the prevention of glutamate-induced neurotoxicity. According to the academic literature, it has been suggested that dietary α-tocotrienol does not reach the brain, including the brains of fetuses and breastfed neonates. Accordingly, it is desirable to identify methods for the efficient enhancement of tocotrienol levels in fetuses and neonates.

Nutritional supplements for adult humans have long contained vitamin E, predominantly in the a-tocopherol form, and occasionally in the tocotrienol form (albeit typically in amounts that fall well below levels that would be clinically beneficial). There is increasing evidence that tocotrienols confer different benefits than α-tocopherol. However, there is also evidence that uptake and maintenance of beneficial levels of tocotrienols in the adult human brain is difficult to achieve. Accordingly, methods and compositions are desirable to enable the efficient uptake of tocotrienols in the adult brain and other critical tissues.

Consumption of a vitamin E deficient diet for only 2 weeks during pregnancy can substantially lower the vitamin E levels of fetal brain while not affecting the vitamin E levels of adult brain, underscoring the importance of proper levels of this vitamin in the diet during pregnancy. When a pregnant mother is provided dietary supplements of tocotrienol, a higher uptake of the α-tocotrienol form of vitamin E by fetal brain is observed as compared to the adult brain. Thus, fetal brain tocotrienol levels are tightly linked to the dietary tocotrienol intake of the mother. In accordance with this disclosure, dietary tocotrienol is bio-available to the brain of a fetus. Not only is tocotrienol delivered to the fetal brain, but gene expression patterns in response to material dietary tocotrienol suggest that vitamin E in the pregnancy diet favorably influences the gene expression profile of the developing fetal brain.

The disclosure provided herein further shows that in adults, uptake of dietary or pharmaceutically supplemented tocotrienol into the adult brain is partially inhibited by tocopherol. It is believed that this differential uptake of these agents is directed by a shared carrier for transport across the blood-brain-barrier. Tocotrienol uptake can be enhanced through the administration of dietary and supplemental forms of tocotrienol compositions that are substantially free of tocopherol.

Dosage and Administration

The tocotrienol composition is administered to subjects who have suffered or at risk of suffering neuronal damage due to trauma, oxidative stress and glutamate toxicity, birth trauma or asphyxia, including adults, pregnant mothers and fetuses, and juveniles. The tocotrienol compositions are administered in an amount sufficient to achieve protection from damage as a result of the effects of glutamate, oxidative stress, and/or 12-LOX mediated damage, preferably before the onset of trauma, and on a continual basis. The tocotrienol compositions are administered to adult subjects in the range from about 1 mg to 1000 mg at a frequency of about 2-3 doses per day. More preferably, dosages are provided to adult subjects in the range from about 100 mg to 800 mg at a frequency of about 2-3 doses per day. Most preferably, dosages of about 600 mg are provided to adult subjects at a frequency of about 2-3 doses per day and are administered in the absence of tocopherol. The preferred form of delivery is gel caps for oral ingestion. Dosages for juvenile subjects are in the range from about 1-1000 mg per dose, and more preferably in the range from about 50-500 mg per dose, and most preferably 300 mg per dose, at a frequency from about 1 to 3 doses per day. Optionally, doses administered to juveniles may lack tocopherol.

Treatment with Tocotrienol to Regain or Enhance Fertility

Numerous studies and products are aimed at the use of vitamin E in the form of tocopherols as part of a nutrition-based regimen of intervention for infertility. However, there is evidence that the effects of tocopherols and other nutritional supplements are not sufficient to inhibit cellular processes that give rise to cell damage. Likewise, certain individuals lack the cellular factors, namely the transport proteins, required for the transport and uptake of tocopherols in tissue such that any beneficial effect of tocopherols is lost in those certain individuals. Thus, the underlying causes of infertility are often not satisfactorily addressed by current nutrition-based and pharmaceutical interventions using the tocopherol form of vitamin E. Accordingly, alternate substances are desirable to provide nutrition-based support for the treatment of infertility.

Tocotrienols may be used in place of or as a supplement to tocopherols in a dietary regimen for the maintenance or resumption of fertility that has been disrupted as a result of to tocopherol mal-absorption or other malfunction in tocopherol uptake or availability. The effects of dietary tocotrienols are long acting in the case of tissues involved in fertility, such as epididymal or abdominal fat or amniotic fluid. Accordingly, an ongoing dietary regimen involving the co-administration of both tocotrienol and tocopherol, is desirable.

Dosage and Administration

The tocotrienol composition is administered to subjects who are experiencing disruption of fertility as a result of to tocopherol mal-absorption or other malfunction in tocopherol uptake or availability. The tocotrienol compositions are administered to adult subjects in the range from about 1 mg to 1000 mg at a frequency of about 2-3 doses per day. More preferably, dosages are provided to adult subjects in the range from about 100 mg to 800 mg at a frequency of about 2-3 doses per day. Most preferably, dosages of about 600 mg are provided to adult subjects at a frequency of about 2-3 doses per day. The preferred form of delivery is gel caps for oral ingestion. Treatments are preferably administered on a daily basis for at least 6 to 8 weeks prior to an intended conception.

Use of Tocotrienol to Permit Breeding of Tocopherol Transport Protein (TTP) Knock-Out Mice

The uptake and transport of dietary or pharmaceutically supplemented tocopherol from the gut to various parts of the body is directed by TTP. In order to understand and distinguish the important roles of tocopherols and tocotrienols in mammals, knock out mice have been produced in which expression of TTP has been ablated. Interruption of TTP results in loss of fertility in these mice which make it impossible to breed and thus maintain the line for further study. The present disclosure provides the first evidence that use of tocotrienol results in the reversal of TTP-dependent infertility in TTP knock-out mice.

Administration and Dosage

The tocotrienol compositions are administered to adult subjects in the range from about 1 mg to 1000 mg at a frequency of about 2-3 doses per day. More preferably, oral dosages are provided to adult subjects in the range from about 1 mg to 500 mg at a frequency of about 2-3 doses per day. Most preferably, dosages of about 50 mg are provided to adult subjects at a frequency of about 2-3 doses per day. Treatments are preferably administered on a daily basis for at least 6 to 8 weeks prior to the intended conception.

Tocotrienol as Reagent in Culture of Brain Cells

Primary neurons are isolated from both adult and juvenile tissue, and have a multitude of uses, including therapeutic and research. The successful culture of primary neurons is of central importance to the viability of clinical programs involving the use of primary neurons implantation and treatment of certain neurodegenerative diseases. Likewise, the establishment and maintenance of primary neurons in culture is essential to the conduct of experiments involving neuronal development, differentiation and response to stimuli. These cells are difficult to maintain in culture due to their ultra-sensitivity to the culture environment. In particular, tissues obtained from more aged subjects are more prone to damage and death in a culture environment.

Tocotrienols have been used effectively for the maintenance of brain cells in primary culture in concentrations from about 0.001 to 100 μM, more preferably in the range from 0.01 to 10 μM, and most preferably in the range from about 0.5 to 2 μM. Good results have been obtained with concentrations of tocotrienols at about 1 μM. Primary neurons are useful for biological studies, for potential diagnostic and therapeutic applications, and for screening drugs. Culture with tocotrienols is particularly useful for neurons from aged subjects since the neuroprotective effects will increase the viability for culture of these otherwise sensitivity cells. Culture with tocotrienols is also useful for stem cells which are intended for use in neuronal applications.

Form and Administration of Tocotrienols

As used herein, the term “biologically effective amount” is an amount sufficient to sufficient to inhibit the activity of 12-LOX. The amount of the tocotrienol required will depend upon the nature and severity of the condition being treated, and on the nature of prior treatments which the subject has undergone and the type of defect or disease being targeted. Ultimately, the dosage will be determined using clinical trials. Initially, the clinician will administer doses that have been derived from animal studies. An effective amount can be achieved by one administration of the tocotrienol composition. Alternatively, an effective amount is achieved by multiple administration of the tocotrienol composition to the subject. The terms “therapeutically effective” and “pharmacologically effective” are intended to qualify the amount of the tocotrienol compositions which will achieve the goal of improvement in disease severity and the frequency of incidence, while avoiding adverse side effects typically associated with alternative therapies. As used herein, the terms “therapeutically effective amount” and “pharmacologically effective amount” mean the total active amount of the tocotrienol compositions that are sufficient to show a meaningful benefit to the subject, i.e., a reduction in disease symptoms associated with neurological trauma or cardiac trauma, or a reduction in tumor size, arrest, inhibition of tumor growth and/or motility or metastasis, and/or an increase in apoptosis, and/or a reduction in the symptoms related to the presence of the tumor, and in the case of infertility, a recovery of the ability to conceive.

The initial dose of the tocotrienol compositions according to the present invention is in the range of 1 mg to 1000 mg at a frequency of about 1 to 3 times per day. While the method of the present relates to the use of the tocotrienols, obviously they may be combined with other therapeutic agents to broaden clinical use. It should be apparent to one skilled in the art that the exact dosage and frequency of administration will depend on the particular compounds employed in the methods of the invention administered, the particular condition being treated, the severity of the condition being treated, the age, weight, general physical condition of the particular patient, and other medication the individual may be taking as is well known to administering physicians who are skilled in this art.

In some embodiments the tocotrienol compositions are substantially free of tocopherols. “Substantially free” as used herein refers to a composition comprising one or more tocotrienols that contains less than 1% by weight of one or more tocopherol compounds. Preferably the composition contains less than 0.5% by weight, more preferably less than 0.1% by weight, and most preferably less than 0.01% by weight of one or more tocopherol compounds.

Compositions containing tocotrienols may be administered via oral, intravenous, intramuscular and intraperitoneal routes. Preferably, the compositions are administered either orally or intravenously, and most preferably, the compositions are administered orally.

It is envisioned that oral administration will be the primary route for preventive and therapeutic administration of the formulations of tocotrienols, although delivery by injection or topical application may also be used. Pharmaceutical compositions containing appropriate dosages of tocotrienols may be prepared with generally used diluents, excipients, vehicles and additives such as filler, extender, binder, carrier, salt, moisturizing agent, disintegrator, disintegrator retarder, absorption promoters, adsorbent, glidant, buffering agent, preservative, dispersing agent, wetting agent, suspending agent, surfactant, lubricant and others. The compositions may have a variety of dosage forms e.g, gel tabs, solution, suspension, emulsion, injection (e.g., solution, suspension).

Solid compositions including tocotrienols in conventional nontoxic solid carriers such as, for example, glucose, sucrose mannitol, sorbitol, lactose, starch, magnesium stearate, cellulose or cellulose derivatives, sodium carbonate and magnesium carbonate. Formulations for topical, i.e., transdermal use include known gels, creams, oils, and ointments. Formulation in a fatty acid source may be used to enhance biocompatibility. Furthermore, the composition may contain coloring agents, preservatives, perfumes, flavors, sweeteners and/or other drugs. Injection, solution, emulsion and suspension forms of the tocotrienols are sterilized and preferably isotonic with blood. Such forms may be prepared using diluents commonly used in the art; for example, water, ethanol, macrogol, propylene glycol, ethoxylated isostearyl alcohol, polyoxyisostearyl alcohol and polyoxyethylene sorbitan fatty acid esters. The compositioins may contain sodium chloride necessary to prepare an isotonic solution, glucose or glycerin, as well as usual solubilizers, buffers and soothing agents.

Capsules, also know as dry filled capsules, are oral solid dosage forms in which the compositions are contained in a swallowable container of suitable size, typically made of gelatin. Hard empty capsules suitable for containing the nutraceutical composition of the present invention are available from several sources, for example, Tishcon Gel-Tec, 2410 N. Zion Rd., Salisbury, Md. 21801; the capsules are supplied in two halves and in various sizes. The sizes are typically designated by number and range from 000 at the larger end of the range and 5 at the smallest end of the range. The capsule halves can be colored by a suitable coloring agent and each halve can be the same or a different color.

Among the dosage forms particularly suitable for the method of this invention are soft gelatin capsules. Thus, from 1 mg to 1000 mg of tocotrienols are mixed with a suitable diluent such as a vegetable oil and then encapsulated in a soft gelatin capsule. Other dosage forms include for example suspensions in which the tocotrienols are suspended or dissolved in alcohol with excipients such as flavoring agents.

Where administered intravenously, suitable carriers include, but are not limited to, physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known in the art.

The inventive compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

### EXAMPLES

The invention may be better understood by reference to the following examples, which serve to illustrate but not to limit the present invention.

### Example 1

### Tocotrienol Formulation # 1 (TOCOMIN® (Manufactured by Carotech Sdn. Bhd.))

1. TOCOMIN 50%

Natural Vitamin E & Tocotrienol Concentrate
Tocomin 50% is a reddish vegetable oil suspension of natural occurring mixture
of tocotrienols and tocopherols, extracted and concentrated from fruits of
palm tree. It contains predominantly of alpha-tocotrienols, gamma-tocotrienols
and delta-tocotrienols. Tocomin 50% also contains natural plant squalene.

| | | |
|---|---|---|
| Total Vitamin E | 50% minimum | |
| | Alpha-tocopherols | 10-14% typical |
| | Alpha-tocotrienols | 10-14% typical |
| | Gamma-tocotrienols | 20-24% typical |
| | Delta-tocotrienols | 3-6% typical |
| Total Palm Squalene | Palm Squalene | 8-12% typical |

| | |
|---|---|
| Isomeric Forms | Approximately 25% typical tocopherols and 75% typical tocotrienols |
| Solubility | Soluble in oils and fats. Insoluble in water. Partially soluble in |
| | ethanol. |
| Peroxide Value | 10 meq/kg max. |
| Moisture | 1.0% max. |
| Microbiology | Total aerobic microbial count - 1000/g max. |
| | Total combined molds & yeasts - 100/g max. |
| Stability | Tocomin 50%'s shelf life is 12 months when stored in cool and dry |
| | place in unopened original containers. |
| Uses | As dietary supplements and nutrients. |
| Packaging | Tocomin 50% is available in 20 kg container under nitrogen. Other |
| | type of packages are available upon request. |
| Labelling | Products formulated using Tocomin 50% can be labelled as containing |
| | “all natural” or “natural-source” vitamin E |
| Storage | Tocomin 50% is sensitive to air, light and heat. Store in tightly closed |
| | containers. |
| Standards | Listed by the FDA as a GRAS nutrient/dietary supplement. |
| Other Ingredients: | Rice Bran Oil, Gelatin, Glycerin, Water, Red Palm Fruit Oil and |
| | Carob (natural color). |
| | Contains no sugar, salt, starch, yeast, wheat, gluten, corn, milk, |
| | preservatives or synthetic Vitamin E. |
| | Tocomin ® is a registered trademark of Carotech Inc. |

### Example 2

### Protection of Rat and HT4 Neurons in Culture

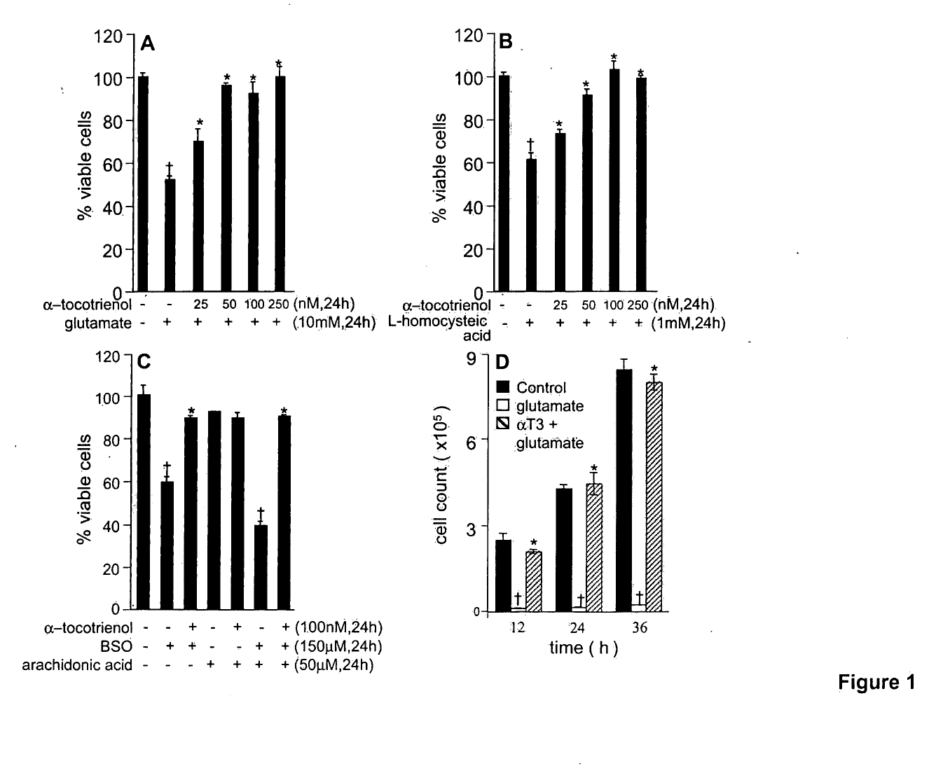

Alpha-tocotrienol protects HT4 neurons from glutamate-induced death at nM concentrations; this protection is independent of α-tocotrienol's antioxidant property (Sen et al., 2000). Referring to FIG. 1, primary rat immature cortical neurons (A-C) or HT4 (D) were either treated or not with α-tocotrienol (as indicated) for 5 min and challenged with either glutamate (10 mM; A); L-homocysteic acid (1 mM; B); or buthionine sulfoximine (0.15 mM; BSO) for 24 h. Arachidonic acid (0.05 mM, C) potentiated BSO-induced cell death. α-Tocotrienol conferred total protection against all of the above neurotoxins. D, 100 nM tocotrienol not only prevented glutamate-induced toxicity but allowed glutamate-treated cells to proliferate at a rate comparable to cells not treated with glutamate. Cells were counted at 12, 24 and 36 h after glutamate challenge. A: †, lower compared to control glutamate non-treated group; *, higher compared to glutamate-treated group. B: †, lower compared to control Lhomocysteic acid non-treated group; *, higher compared to L-homocysteic acid-treated group. C: †, lower compared to corresponding control; *, higher compared to the corresponding group challenged with toxin(s). D: †, lower compared to the corresponding control non-treated group; *, higher compared to the corresponding glutamate-treated group. P<0.05.

In experiments conducted with HT4 and rat neurons, α-tocotrienol at nM concentrations protects immature primary neurons that have been challenged with standard neurotoxins such as glutamate, L-homocysteic acid, L-buthionine-[S,R] sulfoximine (BSO) and a combination of BSO and arachidonic acid (FIG. 1A-C).

Experiments conducted in which HT4 neurons were challenged with glutamate reveal that nM levels of α-tocotrienol not only protect against loss of cell viability but also preserve the normal growth rate of these cells in culture suggesting intact cell function (FIG. 1D).

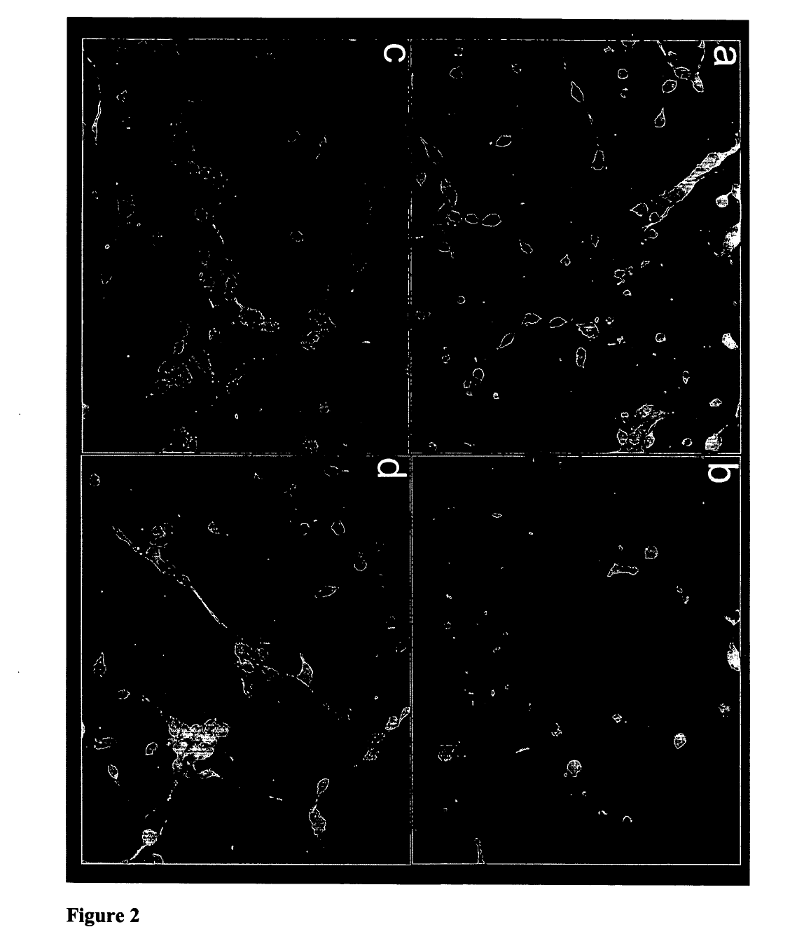

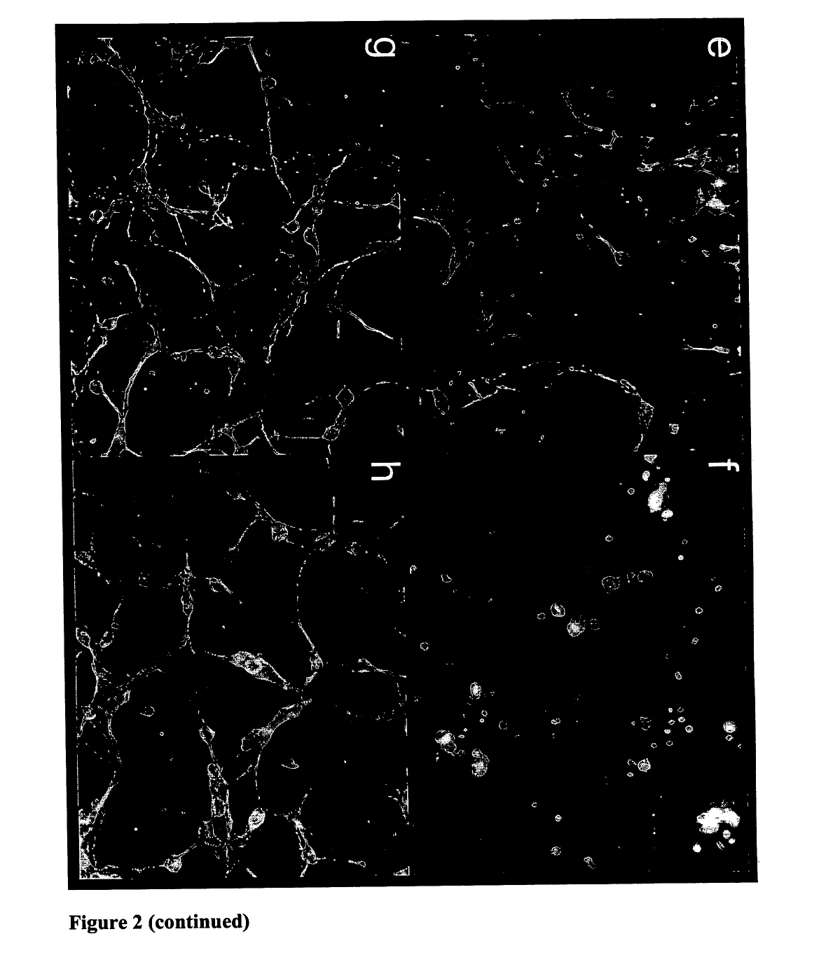

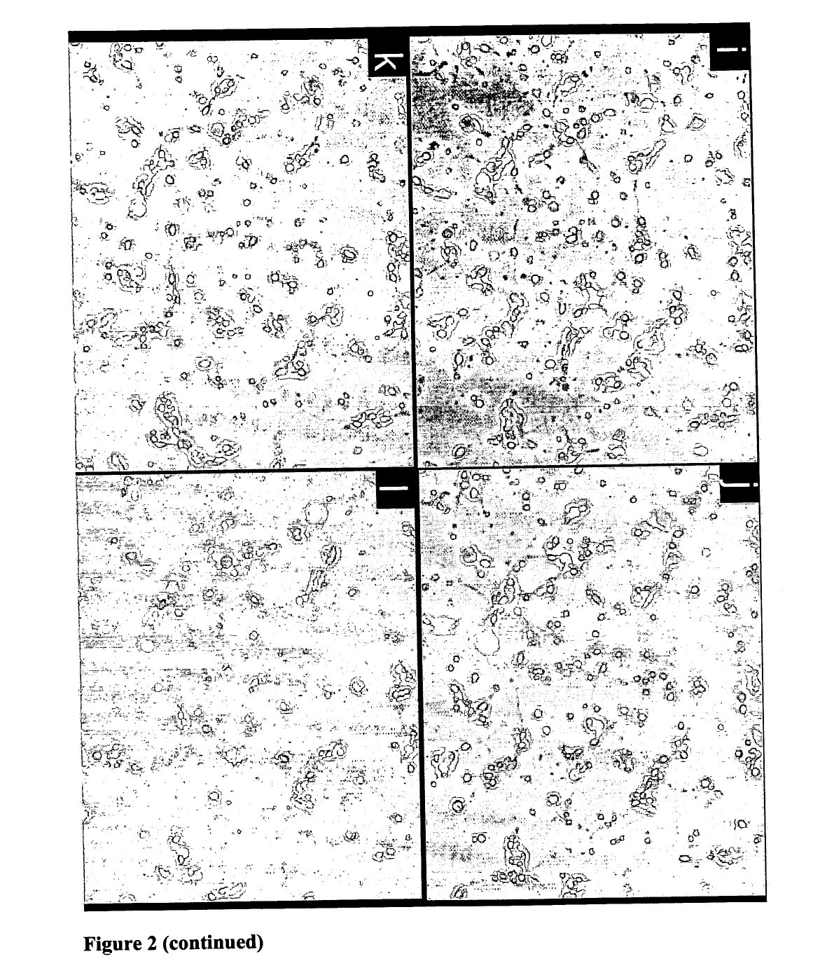

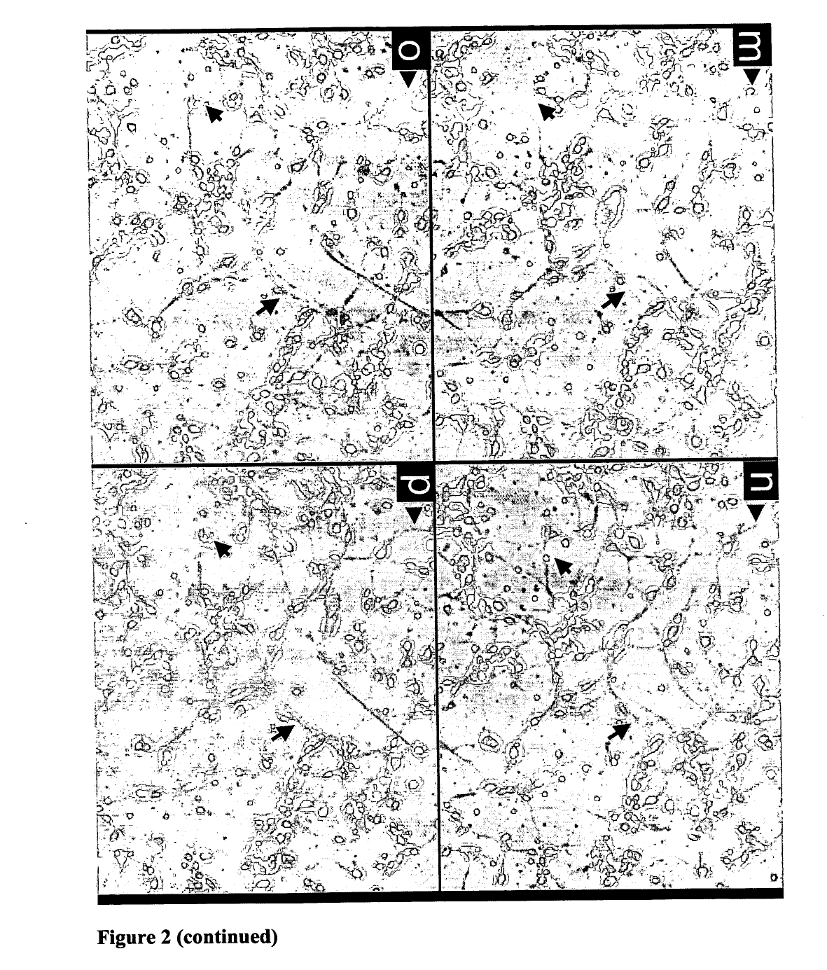

Challenging primary neurons with glutamate results in prominent disruption of the axo-dendritic neural network as evident by the staining of β-tubulin, neurofilament and by time-lapse phase-contrast microscopy. Referring to FIG. 2, after 24 h of seeding, cells were challenged with glutamate. Where indicated, neurons were pre-treated with either atocotrienol (250 nM) or BL15 (2.5 μM) for 5 min prior to glutamate treatment. a-h, Neuron specific Class III α-tubulin in the cultured neural network (for phase contrast microscopy see i-p). After 24 h of glutamate treatment, cells were fixed and stained. a, control; b, glutamate; c, α-tocotrienol +glutamate; d, BL15+glutamate. e-h, Neurofilament staining in the cultured neural network (for phase contrast microscopy see i-p). e, control; f, glutamate; g, α-tocotrienol +glutamate; h, BL15+glutamate. i-p, Live cell imaging of glutamate treated neurons under standard (not glass cover-slip) culture conditions. Phase contrast images were collected once every 15 mins for 18 h from 8 h after glutamate treatment. Frames illustrate time-dependent disintegration of the neural network. i, 8 h; j, 12h; k, 16h; and I, 26h after glutamate treatment. Glutamate-challenged neurons pre-treated with α-tocotrienol (250 nM) resisted degeneration and continued to grow. m, 28h; n, 30h; o, 32h; and p, 34h after glutamate treatment. Two (i-I and m-p) avi video micrographs have been appended for online publication. 200× magnification.

Pre-treatment of cells with α-tocotrienol not only prevents glutamate-induced neuro-degeneration but maintains neuronal growth in the face of 10 mM glutamate (FIG. 2). Protection against glutamate-induced structural alterations in the primary neuron was observed by time-lapse phase-contrast micrography (FIG. 2). Neurons growing in standard culture plates have been successfully images without having to grow them on glass cover slips. Under standard culture conditions neurons and their axo-dendritic network are fairly motile. This is prominently visible in micrographs on tocotrienol treated cells where glutamate was ineffective in triggering neurotoxicity (FIG. 2). Time lapse imaging of glutamate treated control neurons revealed arrest in cytostructural movements before disruption of the network (data not shown).

### Example 3

### Protection of 12-LOX Knockout Mice Neuronal Cells in Culture

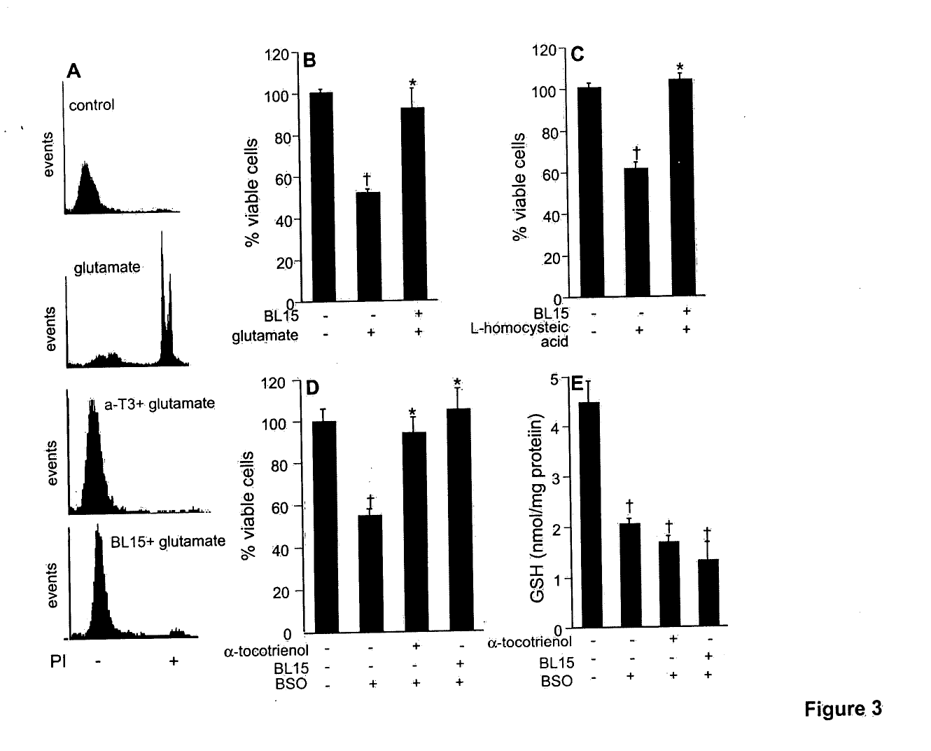

First, we tested for the involvement of 12-LOX in the execution of glutamate-induced death in our model. We started by using the 12-LOX specific inhibitor baicalein or BL15. Referring to FIG. 3, HT4 neurons (A) were either treated or not with a-tocotrienol (250 nM) or BU 5 (2.5 μM, 12-lipoxygenase inhibitor) for 5 min and then challenged with glutamate (10 mM). Cell viability was determined using propidium iodide (PI) exclusion flow cytometry assay. PI−=live; PI+=dead. Rat primary immature cortical neurons (B-D) were either treated or not with α-tocotrienol (100 nM) or BL15 (2.5 μM) for 5 min and challenged either with glutamate (10 mM; B); L-homocysteic acid (1 mM; C) or buthionine sulfoximine (0.15 mM; BSO; D) for 24 h. Arachidonic acid, 50 μM for 24h. BL15, Baicalein 5,6,7-trihydroxy-flavone. Both μ-tocotrienol and BL15 protected neurons against glutamate challenge despite loss of cellular glutathione (GSH; E). B-E: †, lower compared to the corresponding control non-treated group; *, higher compared to the corresponding toxin-treated group. P<0.05.

Pretreatment of cells with BL15 clearly protected against glutamate induced death of HT4 cells as well as that of primary neurons FIGS. 3A and B). In addition, BL15 pretreatment protected primary neurons against toxicity triggered by Lhomocysteic acid or BSO (FIG. 3C&D). Previously we have reported that nM αtocotrienol protects against glutamate-induced death of HT4 cells while not sparing glutamate-induced loss of cellular GSH (27). Comparably, BL15 dependent protection against the toxic effects of glutamate was associated with lowered GSH levels in glutamate-treated primary neurons (FIG. 3E). Although a few key papers have presented pharmacological evidence supporting that glutamate-induced 12-LOX activation plays a significant role in the execution of neuronal death, conclusive evidence is still missing.

### Example 4

### Inhibition of 12-Lipoxygenase by Tocotrienol

Vitamin E and its analogs are known to be potent inhibitors of 5-LOX (37). This effect is independent of the antioxidant property of vitamin E. Vitamin E is also known to inhibit 15-LOX activity by specifically complexing with the enzyme protein (38). A central role of inducible 12-LOX has been proposed in the execution of glutamate-induced neuronal death (16,20). Thus, we sought to examine whether vitamin E αtocotrienol protects glutamateinduced neurodegeneration by inhibiting 12-LOX activity.

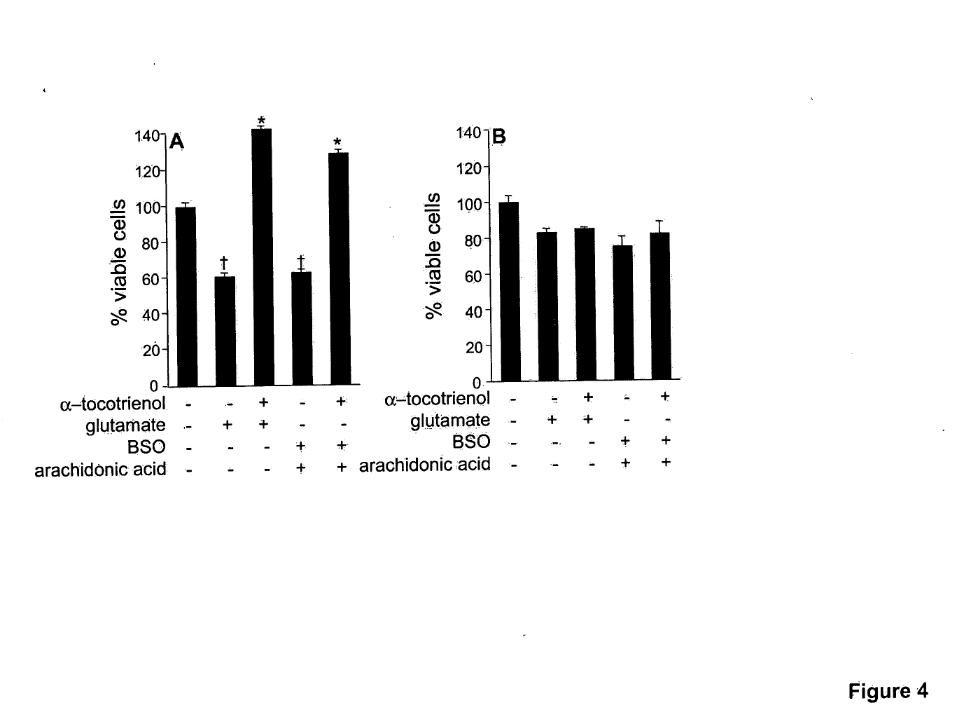

A central role of inducible 12-LOX has been proposed in the execution of glutamate-induced neuronal death (Li et al., 1997; Tan et al., 2001). Referring to FIG. 4, Murine primary immature cortical neuronal cells (C57BL/6, A; B6.129S2-A/ox15^{tmlFun}, B) were challenged with glutamate (10 mM) for 24 h. Cell viability was assessed by lactate dehydrogenase assay. Treatment specifications are described in legend of FIG. 1. α-tocotrienol, 100 nM. †, lower compared to the corresponding control non-treated group, also lower compared to corresponding group in 12-lipoxygenase deficient neurons; *, higher compared to the corresponding toxin-treated group. P<0.05.

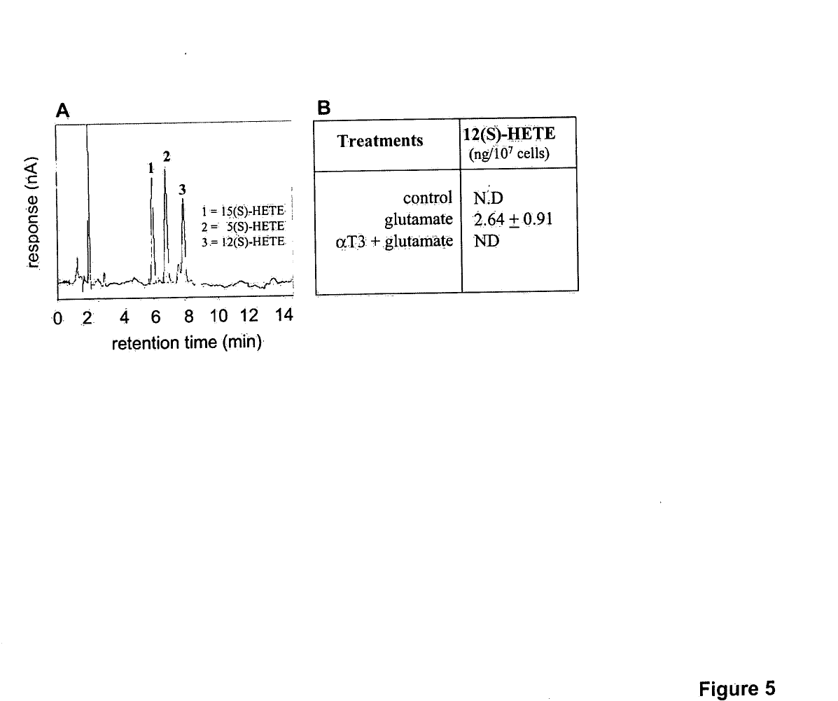

Neurons isolated from 12-LOX deficient mice are resistant to glutamate-induced death (FIG. 4). This striking finding reinforced our interest to test α-tocotrienol as an inhibitor of glutamate-inducible 12-LOX activity in neuronal cells. Referring to FIG. 5, Products of 12-lipoxygenase activity in glutamate-treated neurons were evaluated using a HPLC-based analytical approach. Panel A depicts a representative chromatogram for HETE, a key by-product of lipoxygenase activity; panel B depicts the results of glutamate treatment for 12 h resulted in elevation of 12(S)-HETE levels, a product of 12-lipoxygenase activity, in HT4 neurons. ND, not detectable. It was observed that the by-product of 12-LOX activity, 12(S)-HETE, was not detected in HT4 cells under basal culture conditions (FIG. 5). Glutamate treatment significantly increased cellular 12(S)-HETE content. However, such increase was prevented in α-tocotrienol treated cells (FIG. 5). This line of observation led to the question whether over-expression of 12-LOX in HT4 cells would sensitize them to glutamate-induced cytotoxicity and whether α-tocotrienol could counter such toxicity.

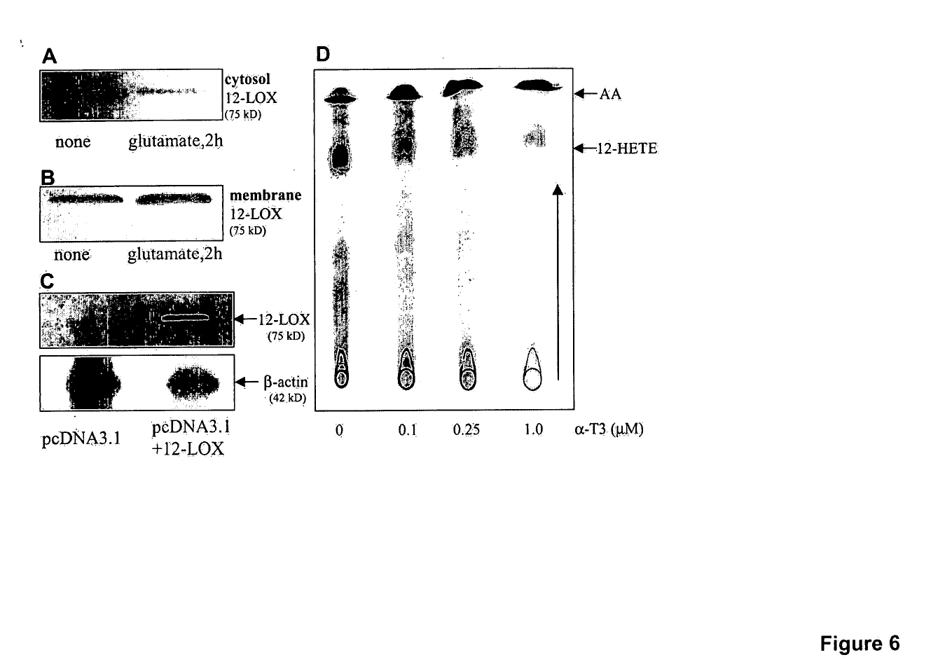

Referring to FIG. 6, HT4 cells were subjected to glutamate treatment for 2 h. Treatment resulted in diminished presence of 12-lipoxygenase in the cytosol (A) and increased presence in the membrane (B) suggesting mobilization of the enzyme from the cytosol to the membrane. Pane C depicts successful over-expression of 12-lipoxygenase in HT4 cells; panel D depicts dose-dependent inhibition of pure 12-lipoxygenase activity by α-tocotrienol. Purified 12-lipoxygenase (porcine leukocyte; 10 units) was incubated with [¹⁴C]-arachidonic acid (25 μM) for 30 min at 37° C. Arachidonic acid and 12-HETE were resolved using thin layer chromatography as described in Materials & Methods We observed that in HT4 cells, treatment with glutamate mobilized cytosolic 12-LOX protein to the cell membrane (FIGS. 6A&B). Thin layer chromatographic analysis of 12-LOX activity in the presence of [¹⁴C]-arachidonic acid revealed that α-tocotrienol dose-dependently inhibited the activity of the pure enzyme (FIG. 6D).

### Example 5

### Three-Dimensional Modeling of 12-LOX

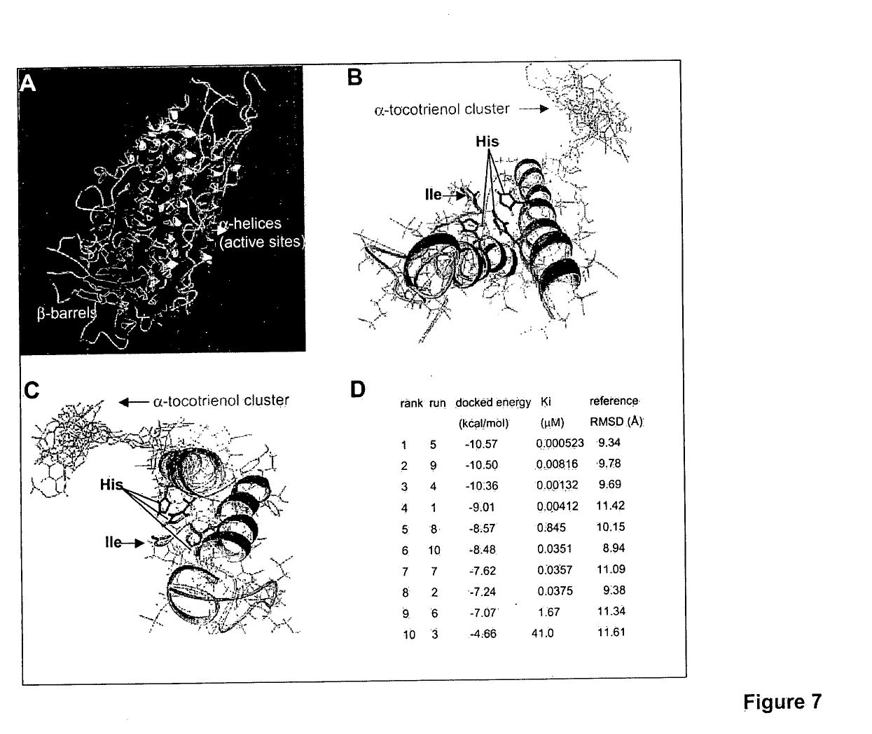

Referring to FIG. 7, three-dimensional modeling of 12-lipoxygenase and α-tocotrienol docking analysis were conducted. A, three-dimensionsal structure of 12-lipoxygenase. Homology model construction was carried out on a Silicon Graphics 02 with 300 MHz MIPS R5000, OS IRIX release 6.5. The theoretical model of 12-lipoxygenase was built using the Sybyl GeneFold module (v6.8, Tripos, Inc., St. Louis, Mo.). B & C, Theoretical model and α-tocotrienol dockings (two positions B & C shown with 10 different docking positions). Amino acid residues in red are His-360, His-365, His-540 and Ile-663 flanking the iron atom can be seen in bold. D, Autodock calculated binding free energies for 10 different docking positions and sorts them in increasing order energy of binding. RMSD, root mean square deviation. The N-terminal domain of lipoxygenases comprises of an eight-stranded antiparallel βbarrel and its molecular size varies with its genomic origin (mammalian or plant) (Minor et al., 1996). The description of size and structure for theoretical model matches the crystal structure of LYGE. In mammalian species, C-terminal of the protein forms catalytic domain of the enzyme and consists of about 18 22 helices and one antiparallel β-barrel sheet. Two long central helices cross at the active site and include histidines for binding the iron ligand (Minor et al., 1996). These histidines were observed in our theoretical model at positions 360, 365 and 540 (FIG. 7A). The terminal isoleucine plays an important role in maintaining the size of active site cavity (Borngraber et al., 1999). The cavity for iron atom active center can also be seen in case of theoretical model. This is the center for dioxygenation reaction and substrate binding (Gillmor et al., 1997). There are 30 solvent cavities that can be observed in theoretical model, with highest cavity of size 124 Cu.A, which is in the vicinity of the active site. The PROCHECK protein geometry for theoretical model calculates 85% of the residues in the allowed region as compared to 88% for 1YGE. The rest of the residues are in the generously allowed region. Autodock calculated binding free energies for 10 different docking positions and sorts them in increasing order energy of binding (FIG. 7D). The docked energy is calculated from the free energy of binding and internal energy of ligand. Inhibition constant is subsequently correlated to the docked energy. We found that α-tocotrienol is concentrated at the opening of a solvent cavity close to the active site (FIG. 7B &C).

Materials and Methods for Examples 2-5.

Materials. The following materials were obtained from the source indicated. L-Glutamic acid monosodium salt; arachidonic acid; dimethyl sulfoxide; L-buthionine-[S,R]sulfoximine; L-homocysteic acid (Sigma St. Louis, Mo.); baicalein; 5,6,7,Trihydroxyflavone (BL15; Oxford Biomedical Research, Oxford, M1); tocotrienol (BASF, Germany; Carotech, Malaysia). For cell culture, Dulbecco's Modified Eagle Medium, Minimum Essential Medium, fetal calf serum and penicillin and streptomycin (Gibco, Gaithersburg, Md.); and culture dishes (Nunc, Denmark) were used.

Cell culture. Mouse hippocampal HT4 cells, were grown in Dulbecco's Modified Eagle Medium supplemented with 10% fetal calf serum, penicillin (100 U/ml) and streptomycin (100 μg/ml) at 37° C. in a humidified atmosphere containing 95% air and 5% CO₂. HT4 cells were provided by Dr. D. E. Koshland Jr. (University of California at Berkeley) (Sen et al., 2000). Primary cortical neurons. Cells were isolated from the cerebral cortex of rat feti (Sprague Dawley; day 17 of gestation) or mouse feti (C57BL/6 mice, day 14 of gestation) as described (Murphy et al., 1990). For 12-LOX knockout studies, neurons were isolated from the feti of B6.129S2-Alox15^{tm1Fun} (Jackson Laboratory, Mich.). After isolation from the brain, cells were counted and seeded in culture plates at a density of 2-3×10⁶ cells per 35 mm plate (Murphy et al., 1990). Cells were grown in Minimal Essential Medium (MEM) supplemented with 10% heat-inactivated fetal bovine serum, 40 μM cystine and antibiotics (100 μg/ml streptomycin, 100 units/ml penicillin, 0.25 μg/ml amphotericin). Cultures were maintained at 37° C. in 5% CO₂ and 95% air in a humidified incubator. All experiments were carried out 24 h after plating.

Treatment with neurotoxic agents. Immediately before experiments, culture media was replaced with fresh medium supplemented with serum and antibiotics. Glutamate (10 mM) was added to the media as aqueous solution (Han et al., 1997a; Sen et al., 2000; Tirosh et al., 2000). No change in medium pH has been observed in response to the addition of glutamate. Other agents used to induce death in neuronal cells have been described in the pertinent figure legends.

Vitamin E treatment. Stock solutions (10³X of working concentration) of α-tocotrienol was prepared in ethanol. Respective controls were treated with equal volume (0.1%, v/v) of ethanol. α-Tocotrienol was added to the culture dishes either 5 min before glutamate, or after the glutamate treatment as indicated in the respective figure legends.

Determination of cell viability. Viability of HT4 cells was determined using propidium iodide exclusion assay using the flow cytometer as described by us previously (Sen et al., 2000; Tirosh et al., 2000). Because primary neuronal cultures tend to aggregate during flow cytometry, the viability of these cells was assessed by measuring lactate dehydrogenase (LDH) leakage (Han et al., 1997a) from cells to media 24 h following glutamate treatment using in vitro toxicology assay kit from Sigma Chemical Co. (St. Louis, Mo., USA). The protocol has been described in detail in a previous report (Han et al., 1997a). In brief, cell viability was determined using the following equation: viability=LDH activity of cells in monolayer/total LDH activity (i.e., LDH activity of cells in monolayer+LDH activity of detached cells+LDH activity in the cell culture media).

12-Lipoxygenase expression. To over-express 12-LOX in HT4, cells were transiently transfected with plasmid pcDNA 3.1 12-LOX (ResGen, Invitrogen Corporation, Carlsbad, Calif.) or pcDNA 3.1 using Fugene 6 (Roche Molecular Biochemical, Indianapolis, Ind.) as per instructions of the manufacturer. To assess the level of 12-LOX expression, HT4 cells were harvested 24 h after transfection and the protein concentrations were determined using BCA protein reagent. Samples (20 μg of protein/lane) were separated on a NuPAGE™ 4-12% Bis-Tris gel (Invitrogen Corporation, Carlsbad, Calif.) under reducing conditions, transferred to PVDF membrane, and probed with 12-LOX polyclonal antiserum (Cayman chemicals, Ann Arbor, Mich.). To evaluate loading efficiency, membranes were stripped and re-probed with anti-βactin antibody (Sigma St. Louis, Mo.).

Cytosol preparation. Cells (1.7×10⁶) were seeded in 140×20 mm plates. After 12-18 h cells were (2×plates per sample) were washed with ice-cold PBS and harvested by scraping from dishes. Samples were spun at 700 g (4° C., 5 minutes). Buffer (400 μl) containing 10 mM HEPES, pH 7.8, 10 mM KCl, 1 mM, EDTA-Na₂, 2 mM MgCl2, 5% glycerol, 1 mM dithiothreitol, 1 mM phenylmethylsulfonyl fluoride, 5 μg/ml leupeptin, 5 μg/ml aprotinin and 5 μg/ml antipain was added to the cell pellet. Samples were resuspened and kept on ice for 15 minutes. After 15 minutes 30 μl of 10% NP40 was added to each sample and samples were vortexed for 30 seconds. This was followed by centrifugation for 20 minutes at 14,000 g at 4° C. The supernatant cytosol was collected and kept in -80° C. The protein concentrations were determined using BCA protein reagent.

Total membrane preparation. Cells (1.7×10⁶) were seeded in 140×20 mm plates. After 12-18 h cells (5 plates per sample) were harvested for total membrane preparation. Total membranes were prepared as described previously (Bashan et al., 1992). After washing with ice-cold PBS, cells were harvested by scraping. Samples were spun at 700 g (4° C., 10 minutes). Buffer (10 ml) containing 20 mM HEPES-Na pH 7.4, 250 mM sucrose, 2 mM, EGTA, 1 mM sodium azide, 100 μM phenylmethylsulfonyl fluoride, 1 μM protease inhibitor cocktail (Sigma St. Louis, Mo.) was added to the cell pellet. Samples were homogenized using motor driven homogenizer (15 strokes) at 4° C. Samples were then spun at 760 g (4° C., 3 minutes). After centrifugation, the supernatant was collected and spun at 190,000 g (4° C., 1 h). The resulting total membrane pellet was resuspened in above-mentioned buffer and samples were stored at −80° C. The protein concentrations were determined using BCA protein reagent.

12-Lipoxygenase activity. To investigate whether tocotrienol directly affect the activity of 12-LOX (12-LOX), 10 units of 12-LOX (Biomol Research labs Inc, Plymouth Meeting, Pa.) was incubated at room temperature for 15 min with or without tocotrienol as indicated in the respective figure legend. The reaction mixture contained 50 mM Tris HCl, pH 7.4 and 1 mM EDTA. After 15 min, the reaction was initiated by adding 25 μM [1-¹⁴C]-arachidonic acid per sample. Samples were kept at 37° C. for 30 minutes. The reaction was terminated by adding 200 μl of ice-cold stop solution containing diethyl ether, methanol and 1 M citric acid at a ratio of 30:4:1 by volume. After mixing, samples were centrifuged and the ethereal extracts were spotted on a silica gel thin layer plate. Thin layer chromatography was performed using a solvent system (diethyl ether, petroleum ether and acetic acid at a ratio of 85:15:0.1 v/v) for 45-60 min at −20° C. Distribution of radioactivity of the substrate and products on the plate were quantified using a imaging analyzer.

Glutathione assay. Glutathione (GSH) was detected using a HPLC-coulometric electrode array detector (Coularray Detector—model 5600 with 12 channels; ESA Inc., Chelmsford, Mass.). Sample preparation, mobile phase and column used for glutathione assay were as previously described (Sen et al., 2000). As an improvement to previously reported methods, the current method implemented a coulometric electrode array detector for the detection of glutathione (Roy et al., 2002). This system uses multiple channels with different redox-potentials. Glutathione was detected at channels set at following potentials: I) 600 mV, II) 700 mV; and II) 800 mV. Signals from channel set at 800 mV were used for quantification (Sen et al., 2002).

12-HETE detection. 12-Hydroxy-eicosatetraenoic acid (HETE) from HT4 cells was detected using a HPLC-UV based method (Eberhard et al., 2000). Immunofluorescence microscopy. For immunofluorescence microscopy, primary cultures of rat cortical neurons were plated on 35 mm plates pre-coated with poly-L-lysine. After 24 h, cell were treated with α-tocotrienol or BL15 for 5 minutes and then challenged with glutamate or exposed to glutamate. After 24 h of glutamate exposure, cells were washed thrice in PBS, fixed for 10 minutes at room temperature in 4% paraformaldehyde, and permeabilized with PBS-T (PBS containing 0.2% Triton X-100) for 20 minutes at room temperature. Samples were then rinsed 3× with PBS-T and blocking (2% BSA in PBS-T) was done for 1 h at room temperature. After blocking, samples were incubated overnight at 4° C. with the primary antibody (anti-neurofilament 200 (1:100, Sigma St. Louis, Mo.) or neuronal class III α-tubulin (1:500, Covance Berkeley, Calif.)}. After washing with PBS (3×, 5 min each), the samples were incubated with Alexa Fluor 488 conjugated goat anti-mouse or anti-rabbit (Molecular Probes Eugene, Oreg.) secondary antibody for 45 minutes at room temperature. This was followed by three PBS washes, and mounting in aqueous medium. Fluorescent images were collected using a Zeiss Axiovert 200M microscope. Images were acquired using Axiovision 3.1.

Live cell imaging. For live cell imaging, primary cultures of rat cortical neurons were plated on 35 mm plates pre-coated with poly-L-lysine. Live cell imaging was performed for non-treated cells from 8h to 26h (18 h duration) of glutamate exposure because that is the time when morphological changes were most prominent. α-Tocotrienol treated cells were insensitive to glutamate. These cells were imaged from 26h-34h (8 h duration) after glutamate treatment to demonstrate healthy growth pattern. Images were collected once every 15 minutes using a specialized phase contrast Zeiss optics suited for imaging cells growing in routine culture plates. The microscope was fitted with appropriate accessories to maintain the stage at 37° C. and the gas environment comparable to that of the culture incubator. Images were exported to avi video format using Axiovision 4.0.

12-Lipoxygenase model. Homology model construction was carried out on a Silicon Graphics O₂ with 300 MHz MIPS R5000, OS IRIX release 6.5. The theoretical model of 12-LOX was built using the Sybyl GeneFold module (v6.8, Tripos, Inc., St. Louis, Mo.). This module employs a BLAST search against the RSCB protein database (http://www.rcsb.org/pdb) to search for possible protein alignments. The module for identifying homologous proteins uses four scoring functions, which include sequence similarity, local interactions, burial similarity and secondary structure similarity. These properties are reflected in combination as an “alignment score”, with a score of 1,000 indicating a perfect alignment with regard to all scoring functions. The target sequence for platelet-type 12-LOX was taken from the NCBI protein database. A BLAST search indicated 97% sequence identity and an “alignment score” of 999.9 with soybean 1-LOX (PDB code 1YGE) (Bernstein et al., 1977), reflecting a similar folding pattern with the target sequence. The structure of LYGE was used subsequently as a template protein for model building using the “backbone method” option in Sybyl. Molecular mechanics calculations were performed using the Tripos force field with a constant dielectric function (ε 2.0) and a non-bonded cutoff distance of 8.0A. The final structure was energy minimized by energy convergence gradient value of 0.05 kcal/mol after assigning the Gasteiger-Hückel charges. The iron atom was then modeled into theoretical model. Protein geometry was checked using PROCHECK (Laskowski, 1993) and was compared to the template protein structure 1YGE.

α-Tocotrienol docking to 12-lipoxygenase. Ligand binding studies were carried out with Autodock (v3.0.5) (Morris, 2001). Autodock is a compilation of three programs, Autotors, Autogrid and Autodock (Goodsell et al., 1996). Autotors facilitates the input of ligand co-ordinates, autogrid pre-calculates a three dimensional grid of interaction energy based on molecular coordinates and autodock performs docking simulations using a Lamarckian Genetic Algorithm. The ligand molecule, a-tocotrienol, was constructed using the Sybyl-Sketch Molecule option, energy minimized and assigned MOPAC charges. Docking was then carried out using standard settings and parameters in AutoDock. Figures for the theoretical model and the dockings were generated using MOLMOL (v2K.2) (MOLecule analysis and MOLecule display) software.

Data presentation. Data shown as bar graphs are mean±SD. Students t test was used to test significance of difference between means. p<0.05 was interpreted as significant difference between means.

### Example 6

### Tocotrienol Protects Cardiac Cells Against Death Induced by Activation of 12-LOX Pathway

Generation of arachidonic acid by the ubiquitously expressed cytosolic phospholipase A(2) (PLA(2)) has a fundamental role in the regulation of cellular homeostasis, inflammation and tumorigenesis. 12-lipoxygenase (12-LO) catalyzes the conversion of arachidonic acid (C20:4) to 12-hydroperoxyeicosatetraenoic acid, which in turn reduces to 12-hydroxyeicosatetraenoic acid (12-HETE) by glutathione peroxidase. We have shown that tocotrienols potently inhibit activation of 12-LOX pathway. Activation of 12-LOX has been implicated in various pathologies of heart. The present work was conducted and demonstrated that tocotrienols can protect cardiac cells against activation of 12-LOX pathway.

Assays were performed using primary cardiac fibroblatsts (CF) isolated from adult (5-6 week old) mouse ventricle. Cells were cultured under 5% O₂ conditions. Five days after isolation cells were treated with arachidonic acid (50 microM)+buthionine sulfoximine in the presence or absence of 250 nM tocotrienol for 24h. Phase contrast imaging was performed using a Zeiss live cell imaging microscope.

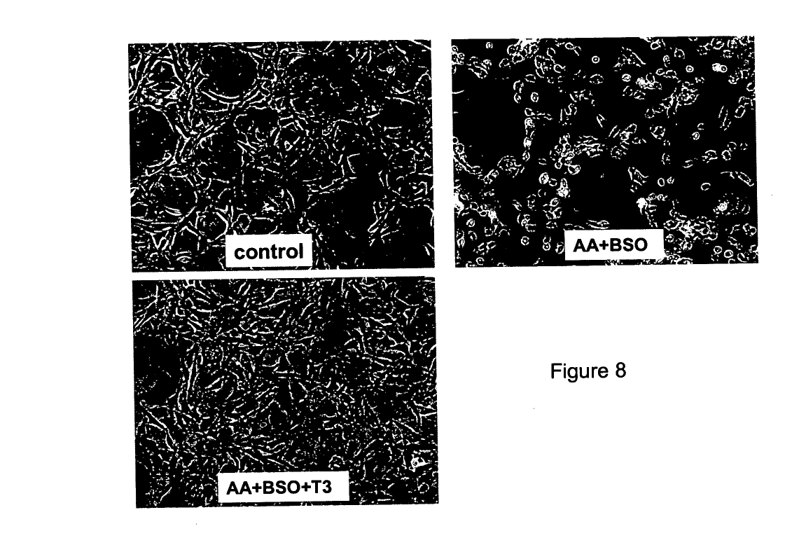

Referring to FIG. 8, isolated cardiac fibroblasts cells were treated five days after isolation with arachidonic acid (AA)(50 microM)±buthionine sulfoximine (BS)) in the presence or absence of 250 nM tocotrienol (T3) for 24 hours. Phase contrast imaging was performed using a Zeiss live cell imaging microscope. Following 24 h, massive cell death was observed in cardiac cells treated with AA, a substrate for 12-LO; and BSO, known to inhibit cellular glutathione synthesis. Lowering of cellular glutathione has been suggested as a trigger for the activation of 12-LOX pathway. Tocotrienol completely blocked cell death induced by AA+BSO (FIG. 8).

### Example 7

### Enhancement of Tocotrienol Concentration in Fetal Rat Brain and Adult Brain

Results disclosed herein provide the first global assessment of vitamin E sensitive genes in a developing fetal brain. Of the 8000 genes surveyed, only 17 genes displayed an increase in gene expression levels in fetal brain as a result of vitamin E feeding to mothers, whereas 34 displayed a decrease in expression indicating that a highly specific set of genes are sensitive to the vitamin E levels in a developing fetal brain.

Based on symptoms of primary vitamin E deficiency in adults, it has been demonstrated that vitamin E has a central role in maintaining neurological structure and function. However, efforts to systematically evaluate the molecular basis of vitamin E action on the brain are lacking. Our data show that α-tocopherol level in the fetal brain was multi-fold lower than that observed in the mother brain. This data is in accordance with a previous study where α-tocopherol levels in fetal brain were lower compared to that of the brains of 21-day-old rats. Furthermore, in humans, the serum α-tocopherol levels in full-term neonates are known to be several folds lower (0.212±0.127 vs. 1.160±0.513 mg/dl) compared to that of their mothers. We have previously shown that compared to α-tocopherol, tocotrienols are strikingly more potent in protecting neuronal cells against glutamate-induced degeneration. However, in vivo data demonstrating the availability of dietary tocotrienols in the brain was lacking. The present study provides first evidence that dietary supplementation of TRF during pregnancy leads to a significant enrichment of α-tocotrienol in both maternal and fetal brains. Dietary vitamin E is absorbed in the intestine and carried by lipoproteins to the liver. In the liver, the α-tocopherol fraction is incorporated into very low-density lipoprotein (VLDL) by a α-tocopherol transfer protein and then secreted into the bloodstream. A recent study shows that scavenger receptor class B type I (SR-BI), which mediates cellular selective cholesteryl ester uptake from lipoproteins, facilitates efficient transfer of α-tocopherol from high-density lipoprotein (HDL) to cultured cells. Furthermore, in SR-BI-deficient mutant mice, relative to wild-type control animals, there was a significant increase in plasma α-tocopherol levels (1.1- to 1.4-fold higher) that was mostly due to the elevated α-tocopherol content of their abnormally large plasma HDL-like particles. Mechanisms of uptake and transport of tocotrienols in organs and tissues are poorly understood in adults and more so in fetal tissues.

HO-3 was one of the few vitamin E sensitive genes up-regulated in fetal brains. HO isozymes, HO-1, HO-2 and HO-3, are heat shock protein 32 protein cognates with a known function of catalyzing the isomer-specific oxidation of the heme molecule, including that of NO synthase. HO-1 is highly inducible, whereas HO-2 and HO-3 are constitutively expressed. These proteins play a central role in the cellular defense mechanisms. HO activity is responsible for the production of equimolar amounts of CO, biliverdin and free Fe. Recent findings with the HOs suggest that these proteins may serve as an intracellular ‘sink’ for NO. LINE1 was identified to be another vitamin E sensitive transcript. The LINE-1, or L1 family of interspersed repeats accounts for at least 10% of the mammalian genome. Like other interspersed repeat DNA families in genomes of other organisms, L1 is dispersed and amplified throughout the genome by a series of duplicative transposition events. Due to the high copy number of L1 sequences in the genome, L1 is abundantly represented in the RNA population of most cells. However, most of the transcripts that contain L1 are the result of fortuitous transcription and are not intermediates in L1 retrotransposition. This high background of L1-containing transcripts, many of which are truncated and rearranged, makes it difficult to distinguish the transcript encoded by an active L1 element(s). ApoB mRNA was one of top candidates that were lower in E⁺ group compared to the E⁻ fetal group. ApoB plays a central role in lipoprotein metabolism and exists in two isoforms in plasma, apoB-100 and apoB-48. High levels of apoB and LDL cholesterol have been associated with an increased risk for coronary heart disease. An earlier study has shown that administration of TRF (100 mg/day) decreases serum apoB. Tocopherol has been shown to inhibit protein kinase C (PKC) activity in cells. PKC-regulated chloride channel was one of the genes that were suppressed in the E⁺ group.

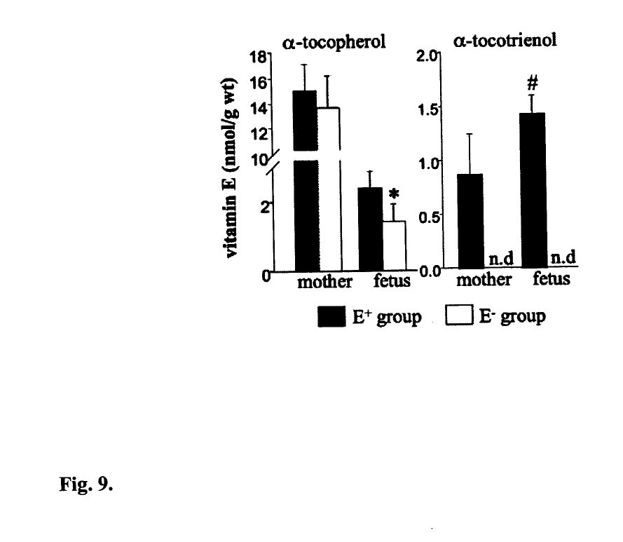

Levles of tocopherol in the brains of maternal and fetal rats were determined. Referring to FIG. 9, pregnant (3 days) rats were randomly divided into (i) E⁺ group—fed a standard rat chow that is enriched in α-tocopherol. Additionally, this group was supplemented for 2 weeks with a daily gavage of TRF suspended in vitamin E-stripped corn oil; and (ii) E⁻ group—fed a vitamin E deficient diet and supplemented with a matched volume of vitamin E-stripped corn oil. On the 17th day of gestation, brains were collected and vitamin E analysis was performed using HPLC. *P<0.05 significantly different compared to the E⁺ group. #P<0.05 significantly different compared to mother brain. n.d., not detected. α-Tocopherol level in the fetal brain was multi-fold lower than that observed in the mother brain (FIG. 9A). Compared to E⁺ group, feeding a vitamin E deficient diet for only 2 weeks during pregnancy did not significantly decrease the α-tocopherol levels in the adult mother brain. However, under similar conditions, feti from the mothers of E⁻ group had significantly lower α-tocopherol levels in brain compared to the feti from E+group (FIG. 9A).

α-Tocotrienol was below detection limits in the brains of mothers as well as feti of the E³¹ group. Oral supplementation of TRF for 2 weeks to mothers during pregnancy resulted in delivery of α-tocotrienol to the mother as well as fetal brains. Importantly, incorporation of tocotrienol in the fetal brain was significantly higher compared to that in the mother brain (FIG. 2B). Of interest, short-term vitamin E deficiency in pregnancy diet did not influence vital parameters of pups such as weight or general health (Applicants' unpublished observations).

### Example 8

### Transcriptome Profiling

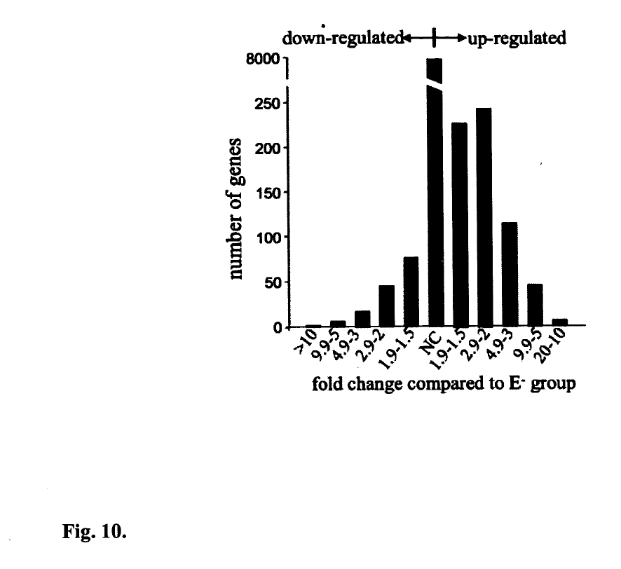

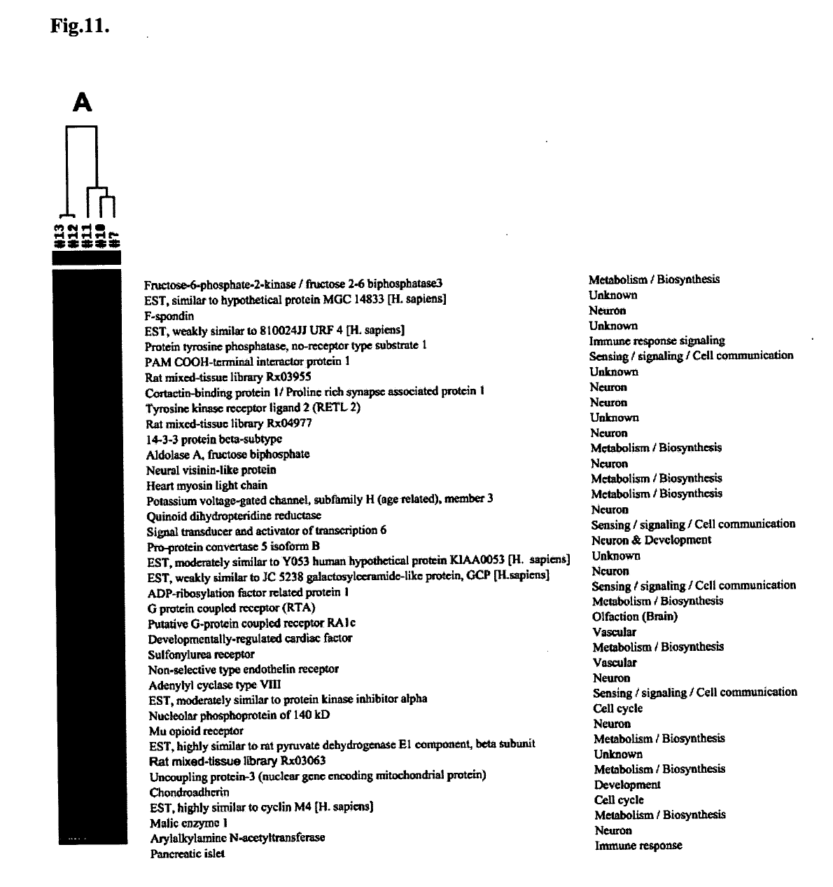

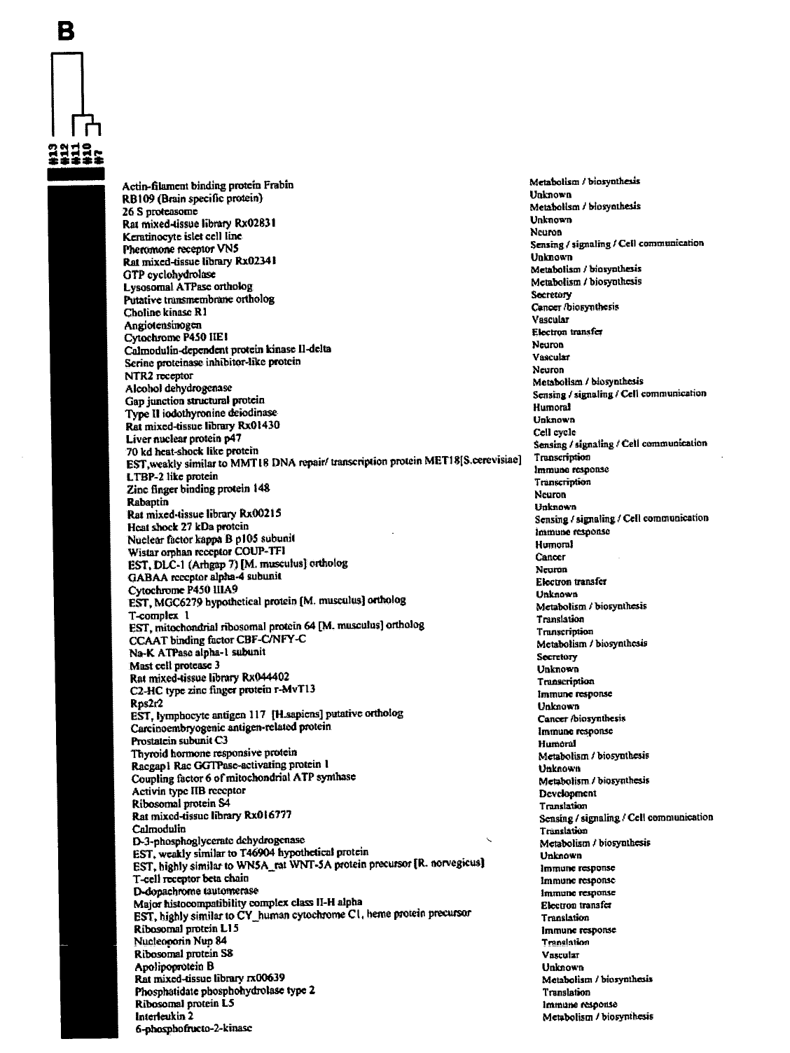

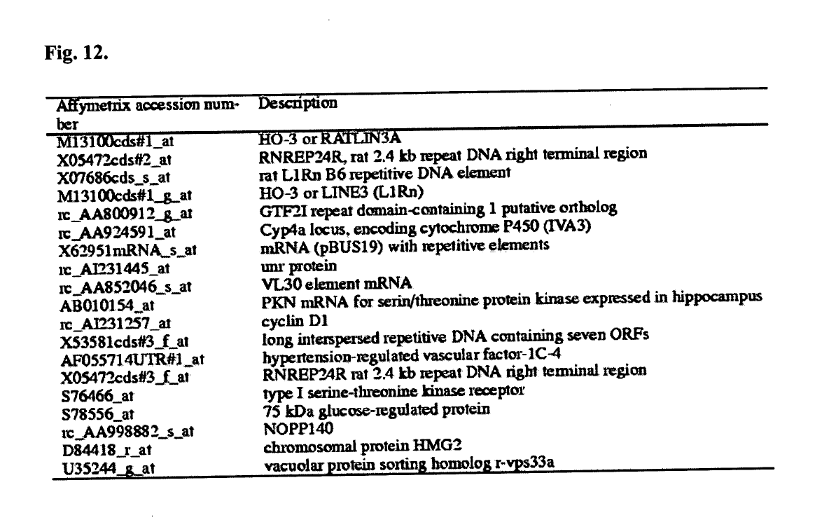

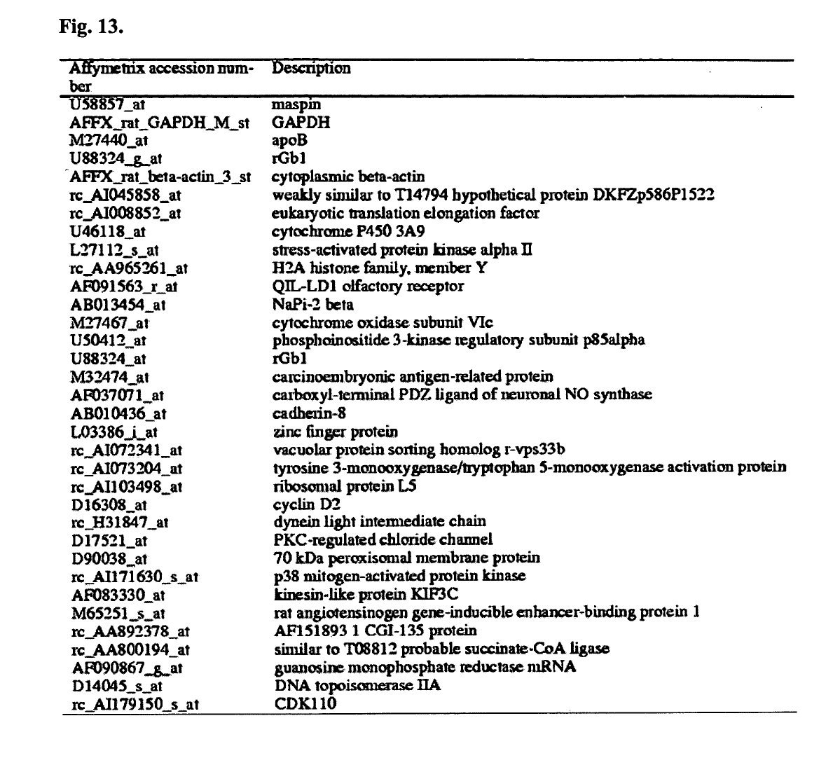

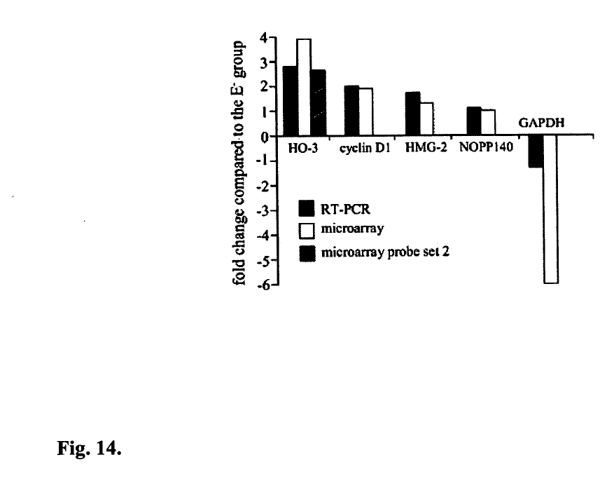

The transcriptomes of developing fetal brains from E⁺ and E⁻ groups were compared using the U34A rat genome high-density oligonucleotide GeneChip array. This array analyzes approximately 7000 full-length sequences and approximately 1000 EST clusters. Using raw data from all replicates available from both groups, a total of six pair-wise comparisons were generated. The average (six pair-wise comparisons) fold-changes of all the genes that were differentially expressed were calculated. Data indicated that a majority of genes remained unchanged (FIG. 10). A total of 645 (7.3%) genes were up-regulated in vitamin E⁺ group compared to the E⁻ group. Out of which 416 genes increased by a magnitude of two-fold or more. On the other hand 152 (1.7%) of the genes were down-regulated with 74 of them lowered by two-fold or more (FIG. 10). Using the t-test analysis described herein, a total 144 genes were observed to have changed significantly (P<0.05) in vitamin E deficiency group compared to the supplemented group. The data was adjusted according to the median center for a clear graphic display of vitamin E sensitive genes (FIG. 11A,B). Next, genes for those the concordance exceeded 50% in pair-wise comparisons were selected, especially if the gene was detected with redundant probe sets. Using this approach of data analysis, a total of 19 probe sets were found to be up-regulated and 34 repressed in E⁺ group compared to E⁻ group (FIG. 12 and FIG. 13). Referring to FIG. 12, six pair-wise comparisons among brains obtained from individual feti, the mothers of whom were fed vitamin E⁺ and vitamin E⁻ diet during pregnancy for 2 weeks. Genes for those the concordance exceeded 50% in pair-wise comparisons were selected, especially if the gene was detected with redundant probe sets. ESTs for which no description is available were excluded. Referring to FIG. 13, six pair-wise comparisons among brains obtained from individual feti, the mothers of whom were fed vitamin E⁺ and vitamin E⁻ diet during pregnancy for 2 weeks. Genes for those the concordance exceeded 50% in pair-wise comparisons were selected, especially if the gene was detected with redundant probe sets. ESTs for which no description is available were excluded.

### Example 9

### Validation of GeneChip Data Using RT-PCR

Select vitamin E sensitive genes identified by the GeneChip approach were verified using conventional semi-quantitative RT-PCR (FIG. 14). Referring to FIG. 14, expression levels of were independently determined using RT-PCR. The following genes identified as differentially expressed in E⁺ group compared to E⁻ group using GeneChip microarray analysis were verified using RT-PCR: HO-3, cyclin D1, HMG2, NOPP140 and GAPDH. The band intensity of the PCR products was quantified and fold change for each gene in E⁺ group compared to E⁻ group was calculated (solid bars). For comparison, fold changes observed in the expression of a specific gene using GeneChip microarray analysis (one or more probe sets) was also plotted (empty and hatched bars). Among the up-regulated genes, two probe sets targeting HO-3 were increased by 3.9 and 3.1 folds, respectively (FIG. 14). In contrast, the expression of maspin, GAPDH, apolipoprotein B (apoB) and G protein beta1 subunit (rGbl) genes was highly (three- to five-fold) repressed in response to dietary vitamin E. The band intensity of the PCR products was quantified and fold change for each gene in E⁺ group compared to E⁻ group was calculated. Data showed that fold change detected using both GeneChip or RT-PCR approaches for genes up-regulated E⁺ vs. E⁻ groups were comparable (FIG. 14). For GAPDH, both microarray as well as RT-PCR data indicated a decrease in expression in E⁺ group compared to E⁻ group. However, the fold-change in GAPDH expression was much higher in the microarray data compared to the RT-PCR data (FIG. 14).

Materials and Methods for Examples 7-9.

Pregnant (3 days) rats (10 weeks old; Sprague-Dawley, Harlan, Indianapolis, Ind., USA) were randomly divided into following two groups: (i) E⁺ group—fed a standard rat chow that is enriched in α-tocopherol (˜200 nmol/g diet). Additionally, this group was supplemented for 2 weeks with a daily gavage of tocotrienol rich fraction (TRF) suspended in vitamin E-stripped corn oil (Harlan). A mixture of 110 mg α-tocopherol and 119 mg of α-tocotrienol contained in 1 g TRF was fed to pregnant rats on a per kg body-weight basis. TRF was provided in the form of Tocomin® 50% provided by Carotech Sdn Bhd (Perak, Malaysia); (ii) E⁻ group—fed a vitamin E deficient diet (TD88163, Harlan; α-tocopherol/tocotrienol levels below detection limits) and supplemented with a matched volume of vitamin E-stripped corn oil. All rats were maintained under standard conditions at 22±2° C. with 12:12 h dark:light cycles. All animal protocols were approved by the Institutional Laboratory Animal Care and Use Committee (ILACUC) of the Ohio State University, Columbus, Ohio, USA. Sample collection. On 17th day of gestation, body weights of each rat were recorded. Rats were killed. Mother and fetal brains were removed, rinsed in ice-cold phosphate-buffered saline, pH 7.4 (PBS) and snap frozen in liquid nitrogen. Samples were briefly stored in −80° C.

Vitamin E extraction and analysis Vitamin E extraction and analysis from mother and fetal brains was performed as described previously using a HPLC-coulometric electrode array detector (Coularray Detector—model 5600 with 12 channels; ESA Inc., Chelnsford, Mass., USA) [13]. This system uses multiple channels with different redox-potentials. α, γ- and δ-tocopherols and tocotrienols were detected on channels set at 200 mV, 300 mV, and 400 mV, respectively.

Affymetrix GeneChip probe array analysis Total RNA was extracted by pulverizing the fetal brains in liquid N₂ followed by extraction using Trizol (Gibco BRL) [14 and 15]. A further clean up of RNA was performed using the RNeasy kit (Qiagen). Targets were prepared for microarray hybridization according to previously described protocols [14]. To assess sample quality the samples were hybridized for 16 h at 45° C. to GeneChip Test-2 arrays. Satisfactory samples were hybridized to Rat Genome arrays (U34A). The arrays were washed, stained with streptavidin-phycoerythrin and were then scanned with the GeneArray scanner (Agilent Technologies) in our own facilities.

Raw data were collected and analyzed using Affymetrix Microarray Suite 4.0 (MAS) and Data Mining Tool 2.0 (DMT) software. The following two approaches were utilized to identify differentially expressed genes: (i) using comparison analysis in MAS, six pair-wise comparisons were generated from replicates of both E⁺ and E⁻ groups. Average fold-changes were calculated for both up- or down-regulated genes. Genes for which the concordance exceeded 50% in pair-wise comparisons were selected, especially if the gene was detected with redundant probe sets; (ii) T-test was performed using DMT, and genes that significantly (P<0.05) changed (increased or decreased) in the E⁺ group compared to the E⁻ group were selected. The average difference values of selected genes were loaded into the Cluster and TreeView software [16]. The data was adjusted according to the median center for a clear graphic display of vitamin E sensitive genes.

Reverse-transcription and polymerase chain reaction (RT-PCR) Expression levels of hemeoxygenase 3 (HO-3), cyclin D1, high-mobility group protein 2 (HMG2), nucleolar phosphoprotein p130 (NOPP140) and glyceraldehyde-3-phosphate dehydrogenase (GAPDH) mRNA were independently determined using RT-PCR as described previously [17]. In brief, the total RNA (1 μg) was transcribed into cDNA using oligo-dT primer and Superscript II. RT-generated cDNA were amplified by PCR using gene-specific primers as described in Table 1. PCR reaction products were electrophoresed in a 1% agarose gel containing 0.25 μg/ml ethidium bromide. The gel was digitally imaged under conditions of ultraviolet transillumination. Quantification of band intensity was performed using the Scion Image (Scion Corporation) that is based on NIH Image software.

TABLE 1
Primers used for RT-PCR

| | |
|---|---|
| mRNA | Primer sequense 5′ to 3′ |

| | |
|---|---|
| Cyclin D1 | CTG CAT GTT CGT GGC CTC TAA GAT |
| | CCA GAA GGG CTT CAA TCT GTT CCT |
| GAPDH | TAT GAC TCT ACC CAC GGC AAG TTC A |
| | CAG TGG ATG CAG GGA TGA TGT TCT |
| HMG2 | TCC TCC CAA AGG TGA TAA GAA AGG A |
| | TGG CAC GGT ATG CAG CAA TA |
| HO-3 | ATG GCA TCA GAG AAG GAA AAC CAT T |
| | CCC ATC AAG TAT TGA GAG CCC ATT C |
| NOPP140 | TCA GTG CCA CCA AGA GTC CCT TAA |
| | CTT CTT CAC TGG AAT CTT CGG AGG A |

### Example 10

### Tocotrienol Formulation #2

| Amount per serving % | | | Daily Value |
|---|---|---|---|
| Vitamin E | 98.8 | iu | 329% |
| Mixed tocopherols | | | |
| Typical distribution: | | | |
| Gamma-tocopherol | 210.0 | mg | |
| Delta-tocopherol | 78.4 | mg | |
| Alpha-tocopherol | 66.3 | mg | |
| Beta-tocopherol | 3.5 | mg | |
| Tocomin ® full-spectrum | | | * |
| natural tocotrienol complex | | | |
| Typical distribution: | | | |
| Gamma-tocotrienol | 35.5 | mg | |
| Alpha-tocotrienol | 18.5 | mg | |
| Delta-tocotrienol | 9.3 | mg | |

The disclosure of all patents, patent applications (and any patents which issue thereon, as well as any corresponding published foreign patent applications), GenBank and other accession numbers and associated data, and publications mentioned throughout this description are hereby incorporated by reference herein. It is expressly not admitted, however, that any of the documents incorporated by reference herein teach or disclose the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While particular embodiments of the subject invention have been described, it will be obvious to those skilled in the art that various changes and modifications of the subject invention can be made without departing from the spirit and scope of the invention. In addition, while the present invention has been described in connection with certain specific embodiments thereof, it is to be understood that this is by way of illustration and not by way of limitation and the scope of the invention is defined by the appended claims which should be construed as broadly as the prior art will permit.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for inhibiting 12-lipoxygenase mediated cytotoxicity in a subject, comprising; administering to the subject a biologically effective amount of tocotrienol.

2. The method of claim 1 wherein the subject is at risk of developing neuronal damage, cardiac tissue damage, integument damage, muscle tissue damage, or combinations thereof.

3. The method of claim 1, wherein the tocotrienol is selected from the group consisting of α-tocotrienol, β-tocotrienol, γ-tocotrienol, δ-tocotrienol, derivatives of these, and combinations of one or more of these.

4. The method of claim 1 wherein the amount of the tocotrienol composition administered on a daily basis is about 600 mg.

5. The method of claim 1 wherein the compositions are substantially free of tocopherol.

6. A method for treating a subject who has suffered from trauma, comprising; administering to said subject a biologically effective amount of tocotrienol.

7. The method of claim 6 wherein the trauma is stroke.

8. The method according to claim 6, wherein the trauma is cardiac trauma.

9. A therapeutic regimen for the prevention or treatment of cancer, comprising administering to a subject in need of the same a pharmacologically effective amount of a pharmaceutical formulation comprising tocotrienol and a pharmaceutically acceptable carrier.

10. The method according to claim 9, wherein the subject is at risk of developing melanoma.

11. A method for protecting neurons in a fetus comprising the step of administering to a pregnant woman who is gestating said fetus a composition comprising at least one tocotrienol.

12. A method for enhancing the concentration of tocotrienol in the brain of a human subject comprising administering to said subject a composition comprising at least one tocotrienol.

13. The method according to claim 12, wherein the composition is substantially free of tocopherol and is administered in the absence of foods or dietary supplements containing tocopherol.

14. The method according to claim 12, wherein said composition is administered at least one half hour after and at least one half hour before said human ingests foods or food supplements containing tocopherol.

15. The method of claim 12 wherein the composition comprises Tocomin®g.

16. The method according to claim 12, wherein the human subject is an infant and the composition is milk or milk extracts obtained from a woman to whom a composition comprising at least one tocotrienol was administered.

17. The method of claim 12, wherein the tocotrienol is selected from the group consisting of α-tocotrienol, β-tocotrienol, γ-tocotrienol, δ-tocotrienol, derivatives of these, and combinations of one or more of these.

18. The method of claim 1-2 wherein the amount of the tocotrienol composition administered on a daily basis is about 600 mg.

19. A method for improving fertility in an animal in need of the same comprising administering to said animal an effective amount of at least one tocotrienol on a daily basis for at least 2 months.

20. The method of claim 19 wherein the amount of the tocotrienol composition administered on a daily basis is about 600 mg.

21. The method of claim 19 wherein expression in the subject of the gene encoding the tocopherol transport protein has been interrupted.

22. A method for maintaining neurons in culture, comprising contacting the neurons with a medium comprising at least one tocotrienol.
